SEQUENCE LISTING (24 sequences)

SEQ ID NO: 1 - DNA, 21 bp, synthetic construct
  source 1..21: mol_type = other DNA
tgtaatgaaagacggcacacc

SEQ ID NO: 2 - DNA, 21 bp, synthetic construct
  source 1..21: mol_type = other DNA
tcttctttgggtattgcttgg

SEQ ID NO: 3 - DNA, 21 bp, synthetic construct
  source 1..21: mol_type = other DNA
ccggagaggagacttcacaga

SEQ ID NO: 4 - DNA, 20 bp, synthetic construct
  source 1..20: mol_type = other DNA
cagaattgccattgcacaac

SEQ ID NO: 5 - DNA, 20 bp, synthetic construct
  source 1..20: mol_type = other DNA
cagcctcttctcattcctgc

SEQ ID NO: 6 - DNA, 20 bp, synthetic construct
  source 1..20: mol_type = other DNA
ggtctgggccatagaactga

SEQ ID NO: 7 - DNA, 22 bp, synthetic construct
  source 1..22: mol_type = other DNA
agcgtcaacagggagatgtcac

SEQ ID NO: 8 - DNA, 23 bp, synthetic construct
  source 1..23: mol_type = other DNA
gggccatatagttccacaaaggc

SEQ ID NO: 9 - DNA, 20 bp, synthetic construct
  source 1..20: mol_type = other DNA
cggactccccacattatcac

SEQ ID NO: 10 - DNA, 20 bp, synthetic construct
  source 1..20: mol_type = other DNA
cacacttgagaccggcattc

SEQ ID NO: 11 - DNA, 21 bp, synthetic construct
  source 1..21: mol_type = other DNA
cttgctgccaccattacgatt

SEQ ID NO: 12 - DNA, 23 bp, synthetic construct
  source 1..23: mol_type = other DNA
tcttcagtccaaattcagatgcc

SEQ ID NO: 13 - DNA, 21 bp, synthetic construct
  source 1..21: mol_type = other DNA
tcacagccgctgaagttactg

SEQ ID NO: 14 - DNA, 19 bp, synthetic construct
  source 1..19: mol_type = other DNA
cagcatgtggtcgtagggg

SEQ ID NO: 15 - DNA, 18 bp, synthetic construct
  source 1..18: mol_type = other DNA
ctgtcatctcccgcctca

SEQ ID NO: 16 - DNA, 19 bp, synthetic construct
  source 1..19: mol_type = other DNA
gctcgagtctccaaagcaa

SEQ ID NO: 17 - DNA, 18 bp, synthetic construct
  source 1..18: mol_type = other DNA
ccagctggtggactctcg

SEQ ID NO: 18 - DNA, 19 bp, synthetic construct
  source 1..19: mol_type = other DNA
gtggcccgagtctagtcct

SEQ ID NO: 19 - DNA, 19 bp, synthetic construct
  source 1..19: mol_type = other DNA
cccggctctttgcctgttt

SEQ ID NO: 20 - DNA, 22 bp, synthetic construct
  source 1..22: mol_type = other DNA
ccagcacctcatagtggttctt

SEQ ID NO: 21 - DNA, 20 bp, synthetic construct
  source 1..20: mol_type = other DNA
tctaccatgagcatcggtgt

SEQ ID NO: 22 - DNA, 21 bp, synthetic construct
  source 1..21: mol_type = other DNA
catcgaagacctcagagccta

SEQ ID NO: 23 - DNA, 20 bp, synthetic construct
  source 1..20: mol_type = other DNA
cggctaccacatccaaggaa

SEQ ID NO: 24 - DNA, 18 bp, synthetic construct
  source 1..18: mol_type = other DNA
gctggaattaccgcggct

(19) United States
(12) Patent Application Publication
(10) Pub. No.: US 2025/0345383 A1
(43) Pub. Date: Nov. 13, 2025

(54) INTRANASAL ADMINISTRATION OF POLYSULFIDE

(71) Applicant: The General Hospital Corporation, Boston, MA (US)

(72) Inventors: Fumito Ichinose, Chestnut Hill, MA (US); Eiki Kanemaru, Cambridge, MA (US)

(21) Appl. No.: 18/867,505

(22) PCT Filed: May 19, 2023
(86) PCT No.: PCT/US2023/022917

§ 371 Date: Nov. 20, 2024

Related U.S. Application Data
(60) Provisional application No. 63/344,095, filed on May 20, 2022.

(51) Int. Cl. A61K 38/06; A61K 9/00; A61K 31/16; A61K 31/385

(52) U.S. Cl. CPC A61K 38/063; A61K 9/0043; A61K 31/16; A61K 31/385

(57) ABSTRACT

Methods and devices for the nasal administration of compositions comprising glutathione trisulfide (GSSSG), pantethine trisulfide (PTN-SSS), or lipoic acid trisulfide (LA-SSS) in neuroprotection, e.g., in neurodegenerative diseases and to reduce the risk of ischemic injury. The methods can be used, e.g., to reduce risk of injury to brain, spinal cord, and peripheral nerves from ischemia or low blood flow states possibly caused by surgery, trauma, and other conditions that decrease/impair blood flow and or oxygen delivery to the nervous system.

### CLAIM OF PRIORITY

This application claims the benefit of U.S. Provisional Application Ser. No. 63/344,095, filed on May 20, 2022. The entire contents of the foregoing are incorporated herein by reference.

### TECHNICAL FIELD

Methods and devices for the nasal administration of compositions comprising glutathione trisulfide (GSSSG), pantethine trisulfide (PTN-SSS), or lipoic acid trisulfide (LA-SSS) in neuroprotection, e.g., in neurodegenerative diseases and to reduce the risk of ischemic injury. The methods can be used, e.g., to reduce risk of injury to brain, spinal cord, and peripheral nerves from ischemia or low blood flow states possibly caused by surgery, trauma, and other conditions that decrease/impair blood flow and or oxygen delivery to the nervous system.

### BACKGROUND

Approximately 2 to 12% of patients who undergo thoracoabdominal aortic surgery experience the devastating complication of paraplegia^{1,2}. More than 80% of the post-surgical paraplegia is reported to be delayed and is caused by secondary neuronal injury in the spinal cord^{3,4}. Although the pathogenetic mechanism of secondary neuronal injury is incompletely understood, increased oxidative stress, mitochondrial dysfunction, inflammation, apoptosis, and glutamate-mediated excitotoxicity have been suggested to play key roles⁵⁻⁶. Because the development of paraplegia is delayed in these patients, there is a window of opportunity for potential preventive intervention. However, no pharmacologic treatment has thus far been shown to mitigate delayed paraplegia after thoracoabdominal aortic surgery.

### SUMMARY

As shown herein, when delivered nasally after reperfusion, glutathione trisulfide (GSSSG) and pantethine trisulfide (PTN-SSS) prevented delayed paraplegia after SCI. The neuroprotective effect of GSSSG was associated with increased local sulfane sulfur concentration in the lumbar spinal cord.

Thus, provided herein are methods of using nasal administration of GSSSG, PTN-SSS, or lipoic acid trisulfide (SSS) for the treatment, or reduction of risk, of a disorder associated with neurodegeneration in a subject. In some embodiments, the methods include comprising preparing the composition comprising GSSSG by dissolving a crystalline form of GSSSG in saline at pH 3-6, e.g, pH 4.8-5.0. Also provided are compositions comprising GSSSG, PTN-SSS, or LA-SSS for nasal administration for use in a method of treatment, or reduction of risk, of a disorder associated with neurodegeneration in a subject, optionally compositions prepared by dissolving a crystalline form of GSSSG in saline at pH 3-6, e.g, pH 4.8-5.0.

In some embodiments, the disorder is post-ischemic neuronal death.

In some embodiments, the disorder is a chronic cerebral degenerative disease, e.g., multi-infarct dementia, Alzheimer's disease, Parkinson's disease, or Lewy body dementia.

In some embodiments, the methods include administering an effective amount of a composition comprising GSSSG, PTN-SSS, or LA-SSS within a few minutes to hours after a traumatic injury occurs.

In some embodiments, the methods include administering an effective amount of a composition comprising GSSSG, PTN-SSS, or LA-SSS before a scheduled thoracic and/or abdominal aortic surgical procedure.

In some embodiments, the methods include administering an effective amount of a composition comprising GSSSG, PTN-SSS, or LA-SSS hours to days before a scheduled thoracic and/or abdominal aortic surgical procedure.

In some embodiments, the methods include administering an effective amount of a composition comprising GSSSG, PTN-SSS, or LA-SSS 2-24 hours, and/or 1, 2, 3, 4, 5, 6, and/or 7 days before the scheduled thoracic and/or abdominal aortic surgical procedure.

Also provided herein are devices for nasal administration of GSSSG, PTN-SSS, or LA-SSS to a subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Chemical structures of polysulfides. A. Chemical structures of GSSSG and GSH. One molecule of GSSSG consists of one molecule of sulfane sulfur (arrow) and two molecules of GSH. B. Chemical structures of PTN-SSS and PTN. One molecule of PTN-SSS consists of one molecule of sulfane sulfur (arrow) and one molecule of PTN. C. Chemical structure of LA-SSS and LA. One molecule of LA-SSS consists of one molecule of sulfane sulfur (red) and one molecule of LA. Abbreviations: GSH, glutathione; GSSSG, glutathione trisulfide; PTN, pantethine; PTN-SSS, pantethine trisulfide. LA-SSS. Lipoic acid trisulfide.

FIGS. 2A-B. A mouse model of spinal cord ischemia-reperfusion injury to investigate the neuroprotective effects of polysulfide. A. A schematic diagram of time course of surgical procedure to produce delayed paraplegia. SCI was induced by clamping the distal aortic arch and the left subclavian artery. B. The protocol for intranasal administration of study drugs after the surgical procedure and restoration of perfusion. The intranasal administration of study drugs was conducted under 3% isoflurane at 0, 8, 23, and 32 hours after surgery. BMS was measured at 0, 8, 24, 48, and 72 hours after surgery to evaluate the hindlimb motor function. Abbreviations: BMS, Basso mouse scale for locomotion; PEEP. positive end-expiratory pressure; RR, respiratory rate; SCI, spinal cord ischemia; TV, tidal volume.

FIG. 3. Post-reperfusion treatment with GSSSG prevented paraplegia after spinal cord ischemia in male and female mice. Changes in BMS scores for 72 hours after SCI in male and female mice subjected to a sham surgical procedure, or SCI and treated with GSSSG, GSH, or vehicle alone. n=11 for each of the GSSSG, GSH, and vehicle alone treatments in the male mouse cohort, and n=8 for each of the GSSSG, GSH, and vehicle alone treatments in the female mouse cohort. n=5 mice for the sham surgical group. Data are presented as means with standard deviation. Abbreviations: BMS, Basso mouse scale for locomotion; GSH, glutathione; GSSSG, glutathione trisulfide; SCI, spinal cord ischemia.

FIGS. 4A-B. Post-reperfusion treatment with GSSSG prevented loss of motoneurons in lumbar spinal cord at 48 hours after spinal cord ischemia. A. Representative photomicrographs of Nissl-stained lumbar spinal cord cross-sections from mice subjected to a sham surgical procedure, or SCI and treated with GSSSG or vehicle alone. An enlargement of the boxed regions is provided below each cross-section. Scale bar=200 μm in low magnification images (40×) and 100 μm in high magnification images (200×). B. Number of viable neurons per one field (0.26 mm²) under high magnification (200×) in the ventral horns of lumbar spinal cord sections from mice subjected to a sham surgical procedure, or SCI and treated with vehicle alone, or GSSSG. Comparisons were made using one-way ANOVA with Tukey's multiple comparison test. n=5 mice for each group. Data are presented as means with standard deviation. Abbreviations: GSSSG, glutathione trisulfide; LSC, lumbar spinal cord; SCI, spinal cord ischemia.

FIGS. 5A-D. Post-reperfusion treatment with GSSSG prevented microglial activation and caspase-3 activation in lumbar spinal cord at 48 hours after spinal cord ischemia. A. Lumbar spinal cords were obtained from mice subjected to a sham surgical procedure, or SCI and treated with GSSSG or vehicle alone. Representative photomicrographs of lumbar spinal cord cross-sections stained with anti-Iba-1 antiserum are shown. An enlargement of the boxed regions is provided below each cross-section. Scale bar=200 μm in low magnification images (40×) and 100 μm in high magnification images (200×). B. Iba-1-positive area per one field (0.26 mm²) under high magnification (200×) in the ventral horns of lumbar spinal cord sections from mice subjected to a sham surgical procedure, or SCI and treated with GSSSG or vehicle alone. Comparisons were made using one-way ANOVA with Tukey's multiple comparison test. The mean value of the Iba-1-positive area in mice in the sham group was set to 1. n=5 mice for each group. Data are presented as means with standard deviation. C. Representative photomicrographs of the ventral horn in lumbar spinal cord cross-sections stained with antibodies that recognize cleaved caspase-3. Scale bar=100 μm. D. Number of neurons with positive cleaved caspase-3 immunoreactivity per one field (0.26 mm²) under high magnification (200×) in the ventral horn of lumbar spinal cord sections. Comparisons were made using Kruskal-Wallis test with Dunn's multiple comparisons test. n=5 mice for each group. Data are presented as medians with an interquartile range. Abbreviations: GSSSG, glutathione trisulfide; Iba-1, ionized calcium binding adaptor molecule 1; LSC, lumbar spinal cord; SCI, spinal cord ischemia.

FIG. 6. Post-reperfusion treatment with GSSSG inhibited upregulation of inflammatory mediators in lumbar spinal cord at 48 hours after spinal cord ischemia. Mice underwent a sham surgical procedure or SCI followed by intranasal administration of GSSSG or vehicle alone. Lumbar spinal cords were harvested and the mRNA levels for each gene were normalized to 18S ribosomal RNA. The mean value of lumbar spinal cord mRNA levels in mice in the sham group was set to 1. n=4-5 mice for each group. *P<0.05, **P<0.01 vs Sham. Data are presented as medians with interquartile range for CCL2, CXCL1, IL-1β, IL-6, and TNF-α, and as means with standard deviation for Bcl-2 and Bcl-XL. Abbreviations: Bcl-2, B-cell lymphoma 2; Bcl-XL, B-cell lymphoma-extra large; CCL2, C-C motif chemokine 2; CXCL1, C-X-C motif chemokine ligand 1; GSSSG, glutathione trisulfide; IL, interleukin; mRNA, messenger RNA; SCI, spinal cord ischemia; TNF-α, tumor necrosis factor-α.

FIGS. 7A-B. Intranasal administration of GS³⁴SSG increased levels of ³⁴S-labeled GSSSG and ratios of ³⁴S-labeled sulfane sulfur species to endogenous sulfane sulfur species in CNS. A. Amount of ³⁴S-labeled GSSSG detected in OB+FB, BS, C+ThSC, and LSC at 30 minutes after intranasal administration of 50 mg/kg of GS³⁴SSG. n=4 mice for OB+FB, BS, and C+ThSC. n=3 mice for LSC. Data are presented as means with standard deviation. B. Ratios of [³²S₂, ³⁴S]GSSH to [³²S₂]GSSH, [³²S, ³⁴S]CysSSH to [³²S₂]CysSSH, and [³²S₂, ³⁴S]CysSSSCys to [³²S₃]CysSSSCys in OB+FB, BS, C+ThSC, and LSC at 30 minutes after intranasal administration of 50 mg/kg of GS³⁴SSG. n=4 mice for each organ. Data are presented as means with standard deviation for the ratios of [³S, ³⁴S]GSSH to GSSH and [³²S₂, ³⁴S]CysSSSCys to [³²S₃]CysSSSCys, and as medians with interquartile range for the ratio of [³²S, ³⁴S]CysSSH to [³²S₂]CysSSH. Abbreviations: BS, brainstem; CNS, central nervous system; C+ThSC, cervical and thoracic spinal cord; CysSSH, cysteine hydropersulfide; CysSSSCys, cysteine trisulfide; GSSH, glutathione hydropersulfide; GSSSG, glutathione trisulfide; LC-MS/MS, Liquid chromatography-tandem mass spectrometry; LSC, lumbar spinal cord; OB+FB, olfactory bulb and forebrain.

FIGS. 8A-B. GSSSG increased relative sulfane sulfur levels in lumbar spinal cord and primary cortical neurons. A. Relative sulfane sulfur levels in lumbar spinal cords at 48 hours after surgery in mice subjected to a sham surgical procedure, or SCI and treated with GSSSG or vehicle alone. Relative sulfane sulfur levels in lumbar spinal cords were estimated using a fluorescent probe, SSip-1. n=6 mice for each group. Data are presented as means with standard deviation. B. Relative sulfane sulfur levels in primary cortical neurons after incubation with GSSSG at 10 μM, Na₂S₃ at 10 μM, or vehicle alone. Data were analyzed using one-way ANOVA with Dunnett's multiple comparison test. n=10 for GSSSG and vehicle groups. n=5 for Na₂S₃ group. Data are presented as means with standard deviation. Abbreviations: GSSSG, glutathione trisulfide; Na₂S₃, sodium trisulfide; SCI, spinal cord ischemia.

FIGS. 9A-C. GSSSG, but not GSH or GSSG, improved cell viability after oxygen and glucose deprivation/reoxygenation. A. Cell viability assessed by crystal violet assay after OGD/R when primary cortical neurons were incubated with GSSSG at various doses. Comparisons were made using one-way ANOVA with Dunnett's multiple comparison test. n=10 for each group. Data are presented as means with standard deviation. B. Cell viability assessed by LDH assay after OGD/R when primary cortical neurons were incubated with GSSSG at various doses. Comparisons were made using one-way ANOVA with Dunnett's multiple comparison test. n=10 for each group. Data are presented as means with standard deviation. C. The cell viability after OGD/R was assessed by crystal violet assay when primary cortical neurons were incubated with GSSSG at 30 μM, GSH at 60 μM, GSSG at 30 μM, or vehicle alone. Comparisons were made using Kruskal-Wallis test with Dunn's multiple comparisons test. n=10 for each group. Data are presented as medians with an interquartile range. Abbreviations: CV, crystal violet; GSH, glutathione; GSSG, glutathione disulfide; GSSSG, glutathione trisulfide; LDH, lactate dehydrogenase; OGD/R, oxygen and glucose deprivation/reoxygenation.

FIGS. 10A-D. PTN-SSS improved cell viability after OGD/R and prevented delayed paraplegia after SCI as a post-reperfusion treatment. A. Cell viability was assessed using the crystal violet assay after OGD/R when SH-SY5Y cells were incubated with PTN-SSS at 5 μM, 10 μM, 25 μM, 50 μM, 100 μM, or vehicle alone. Comparisons were made using one-way ANOVA with Dunnett's multiple comparison test. n=12 for control, vehicle alone and PTN-SSS at 5 μM, 10 μM, and 25 μM. n=6 for PTN-SSS at 50 μM and 100 μM. Data are presented as means with standard deviation. B. Relative sulfane sulfur levels in SH-SY5Y cells after incubation with PTN-SSS at 5 μM, 10 μM, 50 μM, 100 μM, 300 μM, or vehicle alone. Comparisons were made using one-way ANOVA with Dunnett's multiple comparison test. n=6 for each group. Data are presented as means with standard deviation. C. Relative sulfane sulfur levels in the olfactory bulb and forebrain and the whole spinal cord at 30 minutes after intranasal administration of PTN-SSS. Relative sulfane sulfur levels were estimated using a fluorescent probe, SSip-1. Comparisons were made using unpaired t test. n=5 mice for each group. Data are presented as means with standard deviation. D. Changes of the BMS scores for 72 hours after SCI in male mice subjected to SCI and treated with vehicle alone, PTN, or PTN-SSS. Comparisons were made using the Kruskal-Wallis test with Dunn's multiple comparisons test. n=9 mice for each group. Data are presented as means with standard deviation. Abbreviations: BMS, Basso mouse scale for locomotion; CV, crystal violet; LDH, lactate dehydrogenase; OB+FB, olfactory bulb and forebrain; OGD/R, oxygen and glucose deprivation/reoxygenation; PTN, pantethine; PTN-SSS, pantethine trisulfide; SCI, spinal cord ischemia.

FIG. 11. Comparison of the neuroprotective effect of GSSSG and GSSG on hindlimb motor function after SCI. Changes in BMS scores for 72 hours after SCI in male mice subjected to SCI and treated with GSSSG or GSSG. n=6 mice for each group. Data are presented as means with standard deviation. Abbreviations: BMS, Basso mouse scale for locomotion; GSSG, glutathione disulfide; GSSSG, glutathione trisulfide; SCI, spinal cord ischemia.

FIG. 12. Relative mRNA levels of enzymes which synthesize or metabolize sulfide or persulfide in lumbar spinal cords obtained 48 hours after surgery. Mice underwent a sham surgical procedure or SCI followed by intranasal administration of GSSSG or vehicle alone. Lumbar spinal cords were harvested and the mRNA levels for each gene were normalized to 18S ribosomal RNA. The mean value of lumbar spinal cord mRNA levels in mice in the sham group was set to 1. n=4-5 mice for each group. *P<0.05, ****P<0.0001 vs Sham. Data are presented as medians with an interquartile range for CBS, TST, and SUOX, and as means with standard deviation for CSE, 3-MST, SQOR, ETHE1, CARS1, and CARS2. Abbreviations: CARS, cysteinyl-tRNA synthetase; CBS, cystathionine beta synthase; CSE, cystathionine gamma lyase; ETHE1, ethylmaronic encephalopathy 1; GSSSG, glutathione trisulfide; mRNA, messenger RNA; SCI, spinal cord ischemia; SQOR, sulfide:quinone oxidoreductase; SUOX, sulfite oxidase; 3-MST, 3-mercaptopyruvate sulfurtransferase; TST, thiosulfate sulfurtransferase.

FIG. 13. Cytotoxic effect of GSSSG on cell viability. Primary cortical neurons were incubated with GSSSG at 10 μM, 30 μM, 60 μM, 100 μM, or vehicle alone. Cell viability was assessed using the LDH assay. Compared to vehicle-only treated cells, incubation of GSSSG at 100 M significantly increased LDH release (vehicle alone vs GSSSG (100 μM); 105 [85-110]% vs 149 [133-159]%; P<0.0001). Comparisons were made using Kruskal-Wallis test with Dunn's multiple comparisons test. n=10 for each group. Data are presented as medians with an interquartile range. Abbreviations: LDH, lactate dehydrogenase; GSSSG, glutathione trisulfide.

FIG. 14. Cytotoxic effect of PTN-SSS on cell viability. SH-SY5Y cells were incubated with PTN-SSS at 5 μM, 10 μM, 25 μM, 50 μM, 100 μM, or vehicle alone. Cell viability was assessed using the crystal violet assay. Compared to vehicle-only treated cells, incubation of PTN-SSS at 100 μM significantly decreased cell viability (vehicle alone vs PTN-SSS (100 μM); 1.00±0.25 vs 0.59 z 0.21; P=0.0105). Comparisons were made using one-way ANOVA with Dunnett's multiple comparison test. n=12 for vehicle alone and PTN-SSS at 5 μM, 10 μM, and 25 μM. n=6 for PTN-SSS at 50 μM and 100 μM. Data are presented as means with standard deviation. Abbreviations: CV, crystal violet; PTN-SSS, pantethine trisulfide.

FIGS. 15A-C. Effect of sulfide and polysulfide on cell viability of SH-SY5Y cells and primary cortical neurons incubated with MPP*. (A) SH-SY5Y cells or (B and C) murine primary cortical neurons were incubated with or without MPP⁺ with or without trisulfide compounds at 37° C. for 24 h. Cell viability was measured using the crystal violet assay (n=4 or 5 each). *, **, ***p<0.05, 0.01, 0.001 vs. control without trisulfide treatment.

FIG. 16. Effect of intranasal administration of GSSSG on MPTP-induced neurodegeneration. A representative immunoblot shows the level of tyrosine hydroxylase in the nigrostriatal region of mice 7 days after administration of MPTP or saline with or without Na₂S₃ treatment. Quantification of relative levels of tyrosine hydroxylase was determined using densitometry, comparing levels of tyrosine hydroxylase to vinculin (n=5, 4, 5 mice, respectively). **p<0.01 vs. Saline+Saline. ##p<0.05 vs. MPTP+Saline.

### DETAILED DESCRIPTION

Hydrogen sulfide (H₂S), a colorless gas with a characteristic rotten-egg odor, is an environmental hazard produced by various natural and industrial sources. H₂S is also considered to be a signaling molecule, which plays diverse physiological roles⁷. Many effects of H₂S have been attributed to sulfane sulfur species such as persulfides (RSSH) and polysulfides (RSₙH)⁸. The cytoprotective effects of sulfane sulfur species may be mediated by multiple mechanisms, including antioxidant^{9,10} and anti-inflammatory effects^{10,11} inhibition of lipid peroxidation and ferroptosis by scavenging free radicals¹², and post-translational modifications of proteins^{13,14} Sulfane sulfur species produce post-translational modifications in proteins because the sulfane sulfur (S⁰), a sulfur atom with six valence electrons but no charge, is readily donated to acceptor thiols in target proteins in a process known as persulfidation, which modulates function of target proteins^{13,14} In a previous study, we showed that breathing H₂S prevents delayed paraplegia in mice subjected to transient spinal cord ischemia (SCI). The neuroprotective effects of H₂S appeared to be associated with persulfidation of nuclear factor-kappa B (NF-kB) p65¹⁵.

The mechanism by which systemically administered H₂S donor compounds modulate the concentration of reactive sulfur species in target tissue is poorly defined, in part because of the short half-life of H₂S in blood¹⁶. This knowledge gap, concerning the in vivo pharmacokinetics of sulfides, has hindered the application of sulfide-based therapies to patient care. To permit the future use of polysulfides for the treatment of neurodegenerative diseases, it is essential to determine whether administration of polysulfides modulates local concentrations of sulfane sulfur species in the central nervous system (CNS).

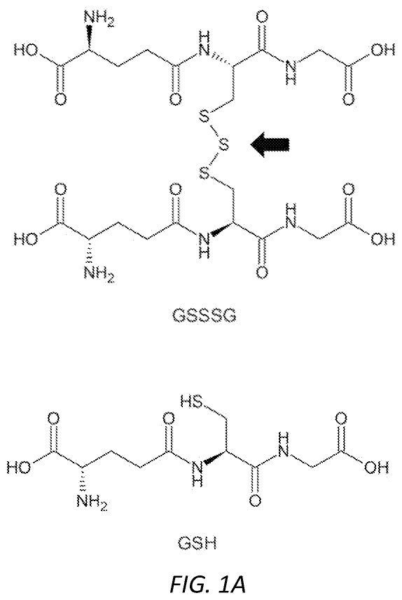

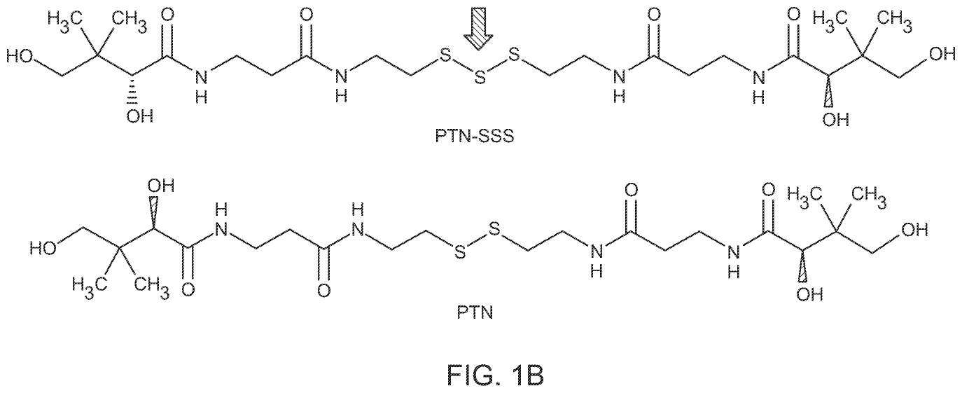

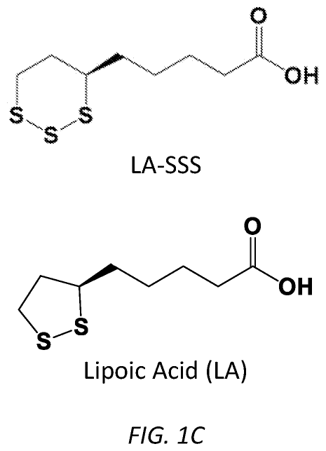

Glutathione trisulfide (GSSSG) is an endogenous polysulfide (FIG. 1A), and is in dynamic equilibrium with various reactive sulfur species including glutathione (GSH), glutathione hydropersulfide (GSSH), glutathione hydropolysulfides, and other glutathione polysulfides^{9,17}. GSSSG may be an important endogenous reservoir of sulfane sulfur species. The observation that the levels of sulfane sulfur species vary in patients, depending on the types and severity of diseases, suggests that sulfane sulfur species may have protective roles in pathological conditions^{18,19}.

GSH, which is a natural tripeptide of glutamate, cysteine, and glycine, is ubiquitous and is the most prevalent thiol (RSH) in mammalian cells. GSH is a nucleophile and acts as a major intracellular antioxidant in mammalian cells²⁰. GSH has been reported to have neuroprotective effects against ischemia-reperfusion injury²¹. Glutathione disulfide (GSSG) is the oxidized form of GSH, and is predominantly produced by GSH peroxidase-mediated catalysis or from the direct reactions of GSH with electrophilic compounds such as radical species²⁰. Both GSH and GSSG can produce post-translational modification of proteins by “glutathionylation”, which protects protein cysteines from irreversible oxidation and regulates the structure and function of a diverse range of proteins^{20,22-24}.

Pantethine (PTN), a precursor for the synthesis of coenzyme A, transfers acetyl groups from pyruvate to oxaloacetate, initiating the tricarboxylic acid cycle²⁵. Preclinical studies suggested beneficial effects of PTN in mouse models of neurodegenerative diseases^{26,27}. Pantethine trisulfide (PTN-SSS), a polysulfide, consists of one molecule of sulfane sulfur and one molecule of PTN (FIG. 1B); see WO2022/045052.

Lipoic acid trisulfides are relatively small molecules (MW=238.39; see, e.g., WO2022/045212) that are expected to pass through the blood brain barrier and be delivered into the CNS. Lipoic acid trisulfides, but not lipoic acid, improved cell viability in a cellular model of Parkinson's disease like GSSSG. After delivery of its “sulfane sulfur” into the cells, the resulting lipoic acid and dihydrolipoic acid may exert their own biological properties, e.g., antioxidant, metal chelator, cofactor for mitochondrial enzymes. LA-SSS consists of one molecule of sulfane sulfur and one molecule of LA. In some embodiments, the LA-SSS is alpha-lipoic acid, or LA-SSS-PCD (lipoic acid trisulfide-beta cyclodextrin or LA-SSS-CE (choline ester); see WO2022/045212.

The present study investigated the neuroprotective effects and pharmacokinetics of intranasal administration of polysulfides in a well-established mouse model of spinal cord ischemia. In this mouse model, neurodegeneration predominantly occurs in the ventral horn of lumbar spinal cord 24-48 hours after reperfusion^{6,28,29}. The experiments determined whether intranasal administration of polysulfides would preferentially increase levels of polysulfides in the CNS³⁰⁻³². It was hypothesized that CNS-targeted, intranasal administration of polysulfides would prevent neurodegeneration in the lumbar spinal cord by increasing the local concentration of sulfane sulfur species and will rescue mice from delayed paraplegia.

In this study, we show that the post-reperfusion intranasal administration of GSSSG, but not GSH or GSSG, prevented the extensive loss of viable neurons in the ventral horns of the lumbar spinal cord and rescued mice from delayed paraplegia after SCI. In primary cortical neurons, GSSSG, but not GSH or GSSG, improved cell viability after OGD/R. The beneficial effects of GSSSG were associated with inhibition of increased levels of inflammatory cytokines and inhibition of microglial- and caspase-3-activation. A marked increase in several ³⁴S-labeled sulfane sulfur species was detected in the lumbar spinal cord shortly after intranasal administration of GS³⁴SSG. Furthermore, we observed that the protective effects of GSSSG were associated with increased sulfane sulfur levels in the lumbar spinal cord after intranasal administration of GSSSG and in primary cortical neurons after incubation with GSSSG. In addition, incubation of SH-SY5Y cells with PTN-SSS increased intracellular sulfane sulfur levels and improved cell viability after OGD/R, and the post-reperfusion intranasal administration of PTN-SSS, but not PTN, rescued mice from delayed paraplegia after SCI. PTN-SSS increased sulfane sulfur levels in the central nervous system shortly after intranasal administration. These observations suggest that sulfane sulfur can be readily delivered to the central nervous system with intranasal administration of polysulfides and that this treatment prevents delayed neurodegeneration in the lumbar spinal cord, mitigating delayed paraplegia. The results of this study underscore the important therapeutic potential of polysulfides in preventing neurodegeneration of the spinal cord.

Previously, we used a chemically-induced cytotoxicity model using SH-SY5Y cells to show that the cytoprotective effects of H₂S-donor compounds were correlated with their ability to increase intracellular sulfane sulfur levels 45. We also reported that the administration of sodium thiosulfate improved the survival and neurological function of mice subjected to global cerebral ischemia-reperfusion; the cytoprotective effects were associated with a marked increase in thiosulfate (a sulfane sulfur species) in plasma and brain tissues⁴⁶. These results suggest that increasing the concentration of sulfane sulfur may be neuroprotective in pathological conditions. In this study, we showed that the neuroprotective effects of intranasal GSSSG in SCI-induced spinal cord injury were associated with increased sulfane sulfur levels in lumbar spinal cords. The results support the hypothesis that the neuroprotective effects of GSSSG are mediated by increased sulfane sulfur.

Previous preclinical studies suggested that administration of GSH ameliorates neuronal cell death after brain ischemia-reperfusion^{21,47}. Because GSH is a metabolic product of GSSSG^{9,17}, it was possible that an increased concentration of GSH could explain the neuroprotective effects of GSSSG. However, in the current study, we observed that intranasal administration of GSSSG, but not GSH, prevented delayed paraplegia after SC. The reason for the discrepancy between the current and previous reports regarding the effects of GSH might arise from differences in doses of GSH, routes of administration, and animal models. In particular, the dose of GSH used in the current study was one-tenth of that used in previous studies. The results of this study support the hypothesis that the mechanism of GSSSG-mediated neuroprotection is independent of conversion to GSH.

Intracellular sulfane sulfur species can react with GSH, resulting in the generation of GSSH⁹. Compared with GSH, GSSH is more nucleophilic and is a better intracellular antioxidant. In addition, GSSH can be directly generated from GSSSG⁹. Akaike and colleagues reported that the concentration of endogenous GSSH in the brain of mice is 222 pmol/mg protein, which is significantly greater than that of other endogenous sulfane sulfur species, including GSSSG (1 pmol/mg protein), CysSSH (2 pmol/mg protein), or CysSSSCys (not detected)^{9,37}. In the current study, ³⁴S-labeled GSSSG was detected at 317±111 pmol/mg protein in the lumbar spinal cord 30 minutes after intranasal administration of GS³⁴SSG. Furthermore, based on the previously reported levels of endogenous GSSH and CysSSH 37, the levels of ³⁴S-labeled GSSH and ³⁴S-labeled CysSSH in lumbar spinal cord after intranasal administration of GS³⁴SSG would have been approximately 1,600 pmol/mg protein and 18 pmol/mg protein, respectively^{9,37}. These results show that intranasal administration of GSSSG can increase the levels of multiple sulfane sulfur species by about 10 to 100-fold in the lumbar spinal cord, the epicenter of neuronal death after SCI. Considering that GSSH is quantitatively the most predominant sulfane sulfur species in lumbar spinal cord after intranasal administration of GSSSG, the bulk of neuroprotective effect of GSSSG might be conferred via GSSH.

Previous studies showed that, after systemic administration, molecules larger than 500 Da were unable to pass through the blood-brain barrier and blood-spinal cord barrier³¹. Because of the large size of GSSSG (644.7 Da), we chose to administer this compound intranasally. After intranasal administration, large molecules can bypass blood-brain- and blood-spinal cord-barriers through the olfactory and trigeminal neural pathways, and can rapidly reach the parenchyma of the central nervous system^{30,32}. The peripheral olfactory system connects the nasal passages with the olfactory bulbs and rostral brain, and the peripheral trigeminal system connects the nasal passages with the brainstem and spinal cord³⁰. For example, a previous report compared the intravenous and intranasal routes of administration on the amount of methylprednisolone sodium succinate (497.5 Da) that reached the spinal cord⁴⁸. While a relatively large amount of methylprednisolone was detected in the parenchyma of the spinal cord after intranasal administration, a much smaller amount of methylprednisolone was detected after intravenous administration⁴⁸. Various other high molecular weight-therapeutics (>500 Da) were successfully delivered to the central nervous system by intranasal administration⁴⁹. In the current study, we observed that the concentration of ³⁴S-labeled GSSSG in the central nervous system (268±140 pmol/mg protein) was markedly higher than that of endogenous GSSSG in the brains of mice (1 pmol/mg protein)³⁷ at 30 minutes after intranasal administration of GS³⁴SSG. In contrast, the concentration of ³⁴S-labeled GSSSG in plasma (25±10 nM) was significantly lower than endogenous GSSSG in the plasma of wild-type mice (125 nM, unpublished data by Akaike and colleagues). These results suggest that intranasally-administered GSSSG readily and preferentially reaches the central nervous system, including the spinal cord, through the olfactory and trigeminal neural pathways, rather than the blood stream.

There are some limitations to this study. First, we did not determine the detailed mechanism responsible for the beneficial effects of polysulfides beyond the fact that the effects are associated with increased levels of sulfane sulfur in the target organ, inhibition of increased levels of inflammatory cytokines, and inhibition of microglial- and caspase-3-activation, and are unlikely to be attributable to transglutathionylation. Polysulfides appear to be cytoprotective via multiple mechanisms including acting as antioxidants^{9,10} and anti-inflammatory agents^{10,11}. inhibiting lipid peroxidation and ferroptosis¹², and enhancing post-translational modifications of proteins^{1,14}. The precise mechanisms responsible for the neuroprotective effects of polysulfides remain to be elucidated in future studies. Second, we did not investigate the mechanism by which GSSSG suppressed the mRNA level of SQOR, a protein that catabolizes sulfides to GSSH. The increased level of GSSH after intranasal administration of GSSSG might downregulate expression of SQOR via negative feedback. Third, although GSSSG and PTN-SSS were administered intranasally under mild sedation, some mice expectorated the drugs from their noses or swallowed them. It is likely that the cytoprotective effects conferred by polysulfides in this study reflect the effects of a lower dose being successfully administered. As better drug formulations, which are more suitable for clinical application, are developed, we anticipate that the beneficial effects of polysulfides will be achieved using lower doses.

The current study revealed that the post-reperfusion intranasal administration of GSSSG or PTN-SSS, but not GSH, GSSG, or PTN, rescues mice subjected to SCI from delayed paraplegia. Intranasally-administered GSSSG accumulated in the lumbar spinal cord, increased the local concentrations of persulfides, polysulfides, and sulfane sulfur, and decreased neuroinflammation, apoptosis, and neurodegeneration. The potent neuroprotective effect of intranasally-administered GSSSG is similar to that of inhaled H₂S, but the use of polysulfides, including GSSSG and PTN-SSS, is far more practical in clinical medicine than administration of gaseous H₂S. In particular, the excellent physical properties of PTN-SSS warrants further evaluation for clinical development. This study opens up the possibility of a novel polysulfide-based therapy to prevent the development of delayed paraplegia after thoracoabdominal aortic surgery and other neurodegenerative diseases of the spinal cord.

We also observed protective effects of trisulfide compounds in MPTP/MPP⁺-induced cell injury, a model of PD. The small size of Na₂S₃ permitted us to deliver the molecule intraperitoneally, with the expectation that it would cross the blood brain barrier. Because of the large size of GSSSG, we delivered this compound intranasally in an animal model of PD, the results showed saw protective effects, demonstrating usefulness in providing neuroprotection in neurodegenerative diseases.

### Methods of Treatment

The methods described herein include methods for the treatment, or reduction of risk, of disorders associated with neurodegeneration in a subject, e.g., a mammalian subject, e.g., a human or non-human veterinary subject. In some embodiments, the disorder is post-ischemic neuronal death, e.g., in the spinal cord. In some embodiments, the disorder is a chronic cerebral degenerative disease (e.g., multi-infarct dementia, Alzheimer's disease, Parkinson's disease, or Lewy body dementia). Generally, the methods include nasal administration of a therapeutically effective amount of a composition comprising a crystalline form of GSSSG, PTN-SSS, or LA-SSS as described herein, to a subject who is in need of, or who has been determined to be in need of, such treatment.

As used in this context, to “treat” means to ameliorate at least one symptom of the disorder associated with neurodegeneration. The conditions that can be treated using a method described herein can be associated with loss of motor control, paralysis or paraplegia. Administration of a therapeutically effective amount of a compound described herein can result in improved motor control, reduced paralysis or paraplegia.

In addition, the methods can result in a reduction in risk of developing loss of motor control, paralysis or paraplegia. Subjects who are at risk of developing loss of motor control, paralysis or paraplegia can include those who have suffered a traumatic injury as well as those who are about to undergo thoracic and/or abdominal aortic surgery. These methods can include nasal administration an effective amount of a GSSSG, PTN-SSS, or LA-SSS composition as described herein within a few minutes to hours after a traumatic injury occurs, and/or before, e.g., hours to days before, a scheduled thoracic and/or abdominal aortic surgical procedure.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. The compositions can be administered one from one or more times per day to one or more times per week; including once every other day. In some embodiments, the GSSSG, PTN-SSS, or LA-SSS is administered every day for at least 2, 3, 4, 5, 6, or 7 days prior to a scheduled thoracic and/or abdominal aortic surgical procedure. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising GSSSG, PTN-SSS, or LA-SSS as an active ingredient. In some embodiments, the compositions are prepared using a crystalline form of GSSSG, using methods described in EP 3560947, by dissolving the crystalline GSSSG in a buffer, e.g., saline, at pH 3-6, e.g., pH 4.8-5. pH can be adjusted with acids or bases, such as hydrochloric acid or sodium hydroxide. Compositions comprising PTN-SSS or LA-SSS can be prepared by dissolving in a buffer, e.g., saline or water, at pH 4-9, e.g., 5-8.

An exemplary method for producing the crystal form of glutathione trisulfide dehydrate can comprise precipitating a crystal of glutathione trisulfide dihydrate in an aqueous solution in which glutathione trisulfide is dissolved, and collecting the precipitated crystal of glutathione trisulfide dihydrate. PTN-SSS or LA-SSS can be prepared as described in WO2022/045212 (LA-SSS) and WO2022/045052 (PTN-SSS).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions for use in the present methods are formulated to be compatible with nasal administration. Examples of routes of administration include parenteral, e.g., intravenous, administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY).

For the purpose of mucosal therapeutic administration, the active compound (e.g., GSSSG, PTN-SSS, or LA-SSS) can be incorporated with excipients or carriers suitable for administration by inhalation or absorption, e.g., via nasal sprays or drops. For nasal administration, the formulations may be an aerosol in a sealed vial or other suitable container.

The pharmaceutical compositions and nasal dosage forms can further comprise one or more compounds that reduce the rate by which an active ingredient will decompose. Thus, the nasal dosage forms described herein can be processed into an immediate release or a sustained release dosage form. Immediate release dosage forms may release the GSSSG, PTN-SSS, or LA-SSS in a fairly short time, for example, within a few minutes to within a few hours. Sustained release dosage forms may release the GSSSG, PTN-SSS, or LA-SSS over a period of several hours, for example, up to 24 hours or longer, if desired. In either case, the delivery can be controlled to be substantially at a certain predetermined rate over the period of delivery.

Nasal delivery is considered an attractive route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions and emulsions can also be delivered. In traditional spray pump systems, antimicrobial preservatives are typically required to maintain microbiological stability in liquid formulations.

Metered spray pumps have dominated the nasal drug delivery market since they were introduced. The pumps typically deliver about 25-200 μL per spray, and they offer high reproducibility of the emitted dose and plume geometry. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination.

Alternative spray systems or devices that avoid the need for preservatives can also be used. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head down application is recommended. Another method used for avoiding preservatives is that the air that replaces the emitted liquid is filtered through an aseptic air filter. In addition, some systems have a ball valve at the tip to prevent contamination of the liquid inside the applicator tip.

For administration by inhalation, the GSSSG, PTN-SSS, or LA-SSS compounds can be delivered in the form of a dry powder or an aerosol spray from pressured container or dispenser that contains a suitable propellant, e.g., a gas such as carbon dioxide, or a nebulizer. Such methods include those described in U.S. Pat. No. 6,468,798.

Devices for nasal administration, e.g., as described herein, comprising GSSSG, PTN-SSS, or LA-SSS are also provided herein.

Described herein are kits that can include a composition comprising GSSSG, PTN-SSS, or LA-SSS, e.g., as an already prepared dry powder or liquid nasal form ready for administration or, alternatively, can include a composition comprising GSSSG, PTN-SSS, or LA-SSS as a solid pharmaceutical composition that can be reconstituted with a solvent to provide a liquid nasal dosage form. When the kit includes GSSSG composition as a solid pharmaceutical composition that can be reconstituted with a solvent to provide a liquid dosage form (e.g., for oral or nasal administration), the kit may optionally include a reconstituting solvent at pH 3-6, e.g., pH 4.8-5.0. When the kit includes PTN-SSS, or LA-SSS composition as a solid pharmaceutical composition that can be reconstituted with a solvent to provide a liquid dosage form (e.g., for oral or nasal administration), the kit may optionally include a reconstituting solvent at pH 4-9, e.g., pH 5-8. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

The pharmaceutical compositions can be included in a container, pack, or dispenser together with instructions for administration. For example, the GSSSG can be provided in a kit in a crystalline form with a sterile buffer (e.g., saline) at pH 3-6 for use in dissolving the crystals to prepare a solution for nasal administration.

### EXAMPLES

The invention is further described in the following examples, which do not limit the scope of the invention described in the claims.

### Materials and Methods

The following materials and methods were used in the Examples below.

### Materials

A stable form of GSSSG dihydrate was synthesized and provided by Kyowa Hakko Bio Co., Ltd. (Tokyo, Japan). GSSSG was suspended in distilled water and dissolved by titrating the pH to between 4.8-5.0 with sodium bicarbonate (Sigma-Aldrich, Saint Louis, MO, USA). To avoid degradation of the compound, a fresh GSSSG solution was prepared immediately before each experiment. GSH (Sigma-Aldrich, Saint Louis, MO, USA) and GSSG hexahydrate (provided by Kyowa Hakko Bio Co., Ltd.) were dissolved in distilled water. In the GSSSG study, the vehicle alone was distilled water, with pH adjusted to between 4.8-5.0 with hydrochloric acid.

PTN-SSS was synthesized and provided by Kyowa Hakko Bio Co., Ltd. The purity of PTN-SSS is 96.3%, and PTN-SSS is highly water soluble (>50 g/L). PTN-SSS and PTN (Toronto Research Chemicals, Toronto, ON, Canada) were suspended in distilled water. PTN-SSS is stable in solution at pH 4.0-9.0 at room temperature for at least 4 days. In the PTN-SSS study, distilled water was used as the vehicle alone.

### In Vivo Studies

### Animals

All animal procedures were performed in accordance with protocols approved by Massachusetts General Hospital Institutional Animal Care and Use Committee and National Research Council's “Guide for the Care and Use of Laboratory Animals”. The study design and the description of experiments followed the ARRIVE guidelines. Adult C57BL/6J mice (12-18 weeks old) of both sexes were purchased from Jackson Laboratory (Bar Harbor, ME, USA). Both male and female mice were used in experiments to investigate the neuroprotective effect of GSSSG on motor function after SCI. Because the neuroprotective effects of GSSSG appeared to be independent of sex, subsequent in vivo experiments were performed in male mice only. All mice were housed in a temperature and humidity-controlled room in the animal center with a 12-hour light/dark cycle, and were provided with food and water ab libitum. To permit access by mice that were recovering from surgery, additional food pellets were inserted into hydrated gel placed on the bedding. A randomized paired design was used to minimize the variability between each treatment group. Mice were paired based on weight, age, delivery dates, and when possible, holding cages. After the pairing, mice were randomly assigned to different treatments.

### Surgery to Induce Spinal Cord Ischemia and Administration of Study Drugs

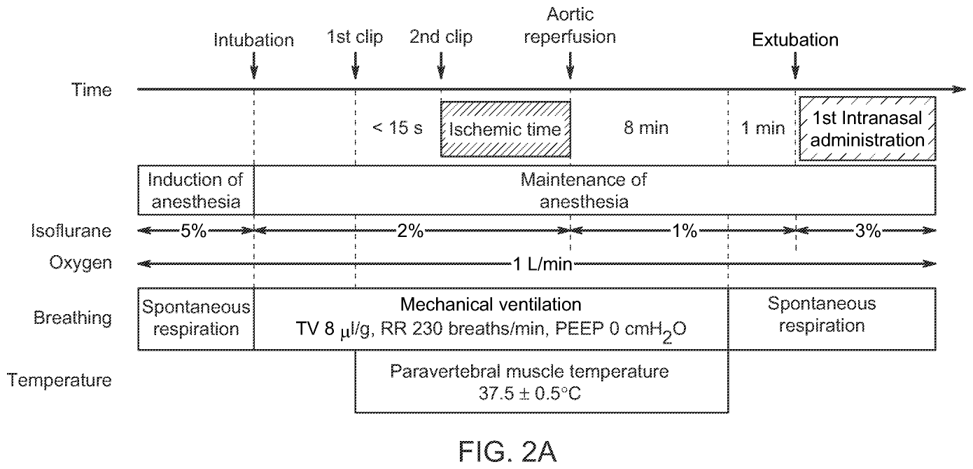

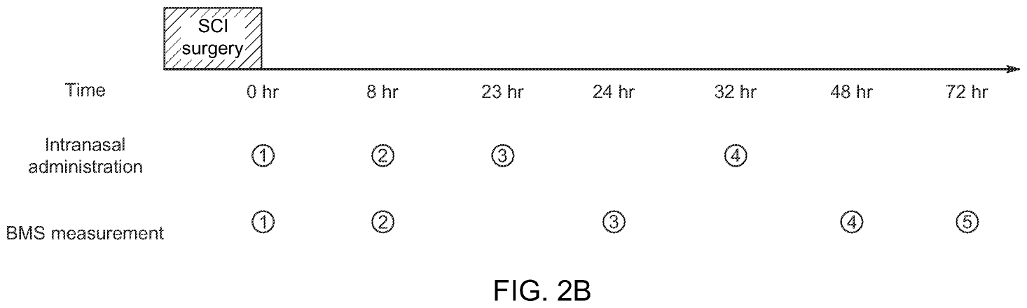

The surgical procedures to produce delayed paraplegia in mice were performed as previously described⁶ (FIG. 2A). Mice were anesthetized with 5% isoflurane in 100% O₂ and tracheally intubated with a 20-gauge catheter (Angiocath; Becton Dickinson, Franklin Lakes, NJ, USA). Mice were mechanically ventilated (MiniVent model 845; Harvard Apparatus, Holliston, MA, USA) and anesthesia was maintained with 2% isoflurane in 100% O₂, with tidal volume 8 μl/g. The paravertebral muscle temperature was measured using a T-type implantable thermocouple probe (IT-18) and a T-type pod (ADInstruments, Colorado Springs, CO, USA). After a skin incision was made on the back, the tip of the probe was placed at the level of L1-L3 using an 18-gauge needle and the temperature was maintained at 37.5±0.5° C. using a heating pad and DC temperature controller (FHC, Bowdoin, ME, USA). A median sternotomy extended from the apex of the manubrium to the second rib. The aortic arch was gently isolated between the left common carotid artery (LCCA) and the left subclavian artery (LSA), avoiding the vagus nerve and the left recurrent laryngeal nerve. A first clip (straight micro clip, RS-5424; Roboz Surgical Instrument Company, Inc., Gaithersburg, MD, USA) was placed on the aortic arch between the LCCA and LSA, and then, within 15 seconds, a second clip (450 angle micro clip, RS-5435; Roboz Surgical Instrument Company, Inc., Gaithersburg, MD, USA) was placed on the origin of the LSA. After ischemia, the clips were removed in reverse order. Incisions were closed in layers and mechanical ventilation was discontinued. After stable spontaneous respiration was confirmed, mice were extubated.

The previously described surgical procedures⁶ were modified as follows. The respiratory rate during the procedure was increased to 230 breaths/minute, because hyperventilation promotes the occurrence of delayed paraplegia caused by spinal cord ischemia³³. A laser Doppler perfusion monitor (moorVMS-LDF1; Moor Instruments, Millwey, UK) was used to monitor the femoral artery blood flow^{28,29}. A plastic fiber (POF500; Moor Instruments, Millwey, UK) was affixed perpendicular to the left femoral artery, to confirm that occlusion of aorta resulted in an immediate and sustained reduction (>90%) in the femoral artery blood flow^{28,29}. To improve the accuracy of surgical procedures, a microscope (Leica MZ95; Leica Microsystems, Buffalo Grove, IL, USA) was used to isolate the aortic arch and place the clips. Based on the results of pilot studies, we selected the ischemic time which induced delayed paraplegia in all mice (3 minutes for male mice, 3.5 minutes for female mice) with the lowest mortality rate at 72 hours after surgery (Table 1).

TABLE 1
Spinal cord ischemic time causing delayed paraplegia for each sex.

| Ischemic | Total number of | Number of mice | Mortality rate at |
|---|---|---|---|
| time | mice subjected to | exhibiting delayed | 72 hours after |
| (minutes) | SCI | paraplegia | surgery (%) |
| Male | | | |
| 2.5 | 5 | 1 | 0 |
| 3 | 7 | 7 | 0 |
| 3.5 | 7 | 7 | 28.5 |
| 4 | 7 | 7 | 42.8 |
| 4.5 | 4 | 4 | 50 |
| 5 | 5 | 5 | 60 |
| Female | | | |
| 3 | 5 | 1 | 0 |
| 3.5 | 5 | 5 | 0 |
| 4 | 5 | 5 | 20 |

Abbreviations: SCI, spinal cord ischemia.

Study drugs were administered intranasally using a single-channel pipettor. Mice were anesthetized with 3% isoflurane using a non-rebreathing circuit with a mouse nose cone (VetEquip, Inc., Livermore, CA, USA). While the drugs were administered, the nose cone was removed and mice were allowed to breathe air. Approximately 6 μL of each drug was administered into the mouse's nostrils and the mouse inhaled the droplets during inspiration. This procedure was repeated at intervals until the total volume of drug was administered, which took approximately 10 minutes. The interruption time of isoflurane administration for each intranasal dose was about 10 seconds. The intranasal drugs were administered 0, 8, 23, and 32 hours after surgery (FIG. 2B). In the sham procedure group, the surgical procedures described above were conducted, but the aorta was not cross-clamped.

For pain control, 0.1 mg/kg of buprenorphine was administered intraperitoneally before surgery and every 12 hours until 60 hours after surgery. We also administered 0.5 mg/kg of 0.25% bupivacaine subcutaneously around the wound incision site immediately after surgery.

Assessment of Motor Function after Spinal Cord Ischemia

The hindlimb motor function was quantified using the Basso mouse scale for locomotion (BMS)³⁴ at 0, 8, 24, 48, and 72 hours after surgery (FIG. 2B). This score ranges from 0 for complete paraplegia to 9 for normal motor function. A BMS score <6 (0-5) indicates paraplegia or paraparesis, whereas a BMS score ≥6 (6-9) indicates that mice are able to walk.

Studies to Investigate the Neuroprotective Effect of Post-Perfusion Treatment with GSSSG on Motor Function after Spinal Cord Ischemia

Mice were randomly assigned to each treatment group and the investigator who performed the surgical procedure was blinded to the group assignment. Based on pilot studies, 50 mg/kg of GSSSG, 45.2 mg/kg of GSH (twice the molar amount of GSSSG because one molecule of GSSSG contains two molecules of GSH) (FIG. 1A), or 53 mg/kg of GSSG (an equimolar dose of GSSSG) was administered.

### Histological Studies

The lumbar enlargement of the spinal cord was removed 48 hours after surgery and sectioned to 5 m thickness using a cryotome (CM1850UV; Leica Biosystems, Heidelberger, Germany). Nissl staining was conducted using the Nissl stain kit (VitroVivo Biotech, Rockville, MD, USA) according to the protocol recommended by the manufacturer. Immunohistochemical staining for ionized calcium-binding adaptor molecule 1 (Iba-1) and cleaved caspase-3 was performed as previously described^{6,15}. The stained sections were examined using an epifluorescence microscope (Nikon Eclipse 80i; Nikon Instruments, Inc., Melville, NY, USA).

The number of stained cells was counted in one field (0.26 mm²) under high magnification (200×) in 3 different sections of spinal cord from each mouse by an investigator who was blinded to the identity of the samples. The average number of stained cells was calculated for each mouse. For quantitative analysis of Iba-1 staining, the Iba-1-positive area per one field (0.26 mm²) under high magnification (200×) in 3 different ventral horn sections for each mouse was calculated using the ImageJ image-processing program (National Institutes of Health, Bethesda, MD, USA). The mean area of Iba-1 staining in the spinal cords of mice in the sham procedure group was set to 1 and the relative amount of Iba-1 staining was determined for each experimental group of mice. Each group included 5 mice.

### Measurements of Gene Expression

Messenger RNA (mRNA) levels were measured as previously described¹⁵ using lumbar spinal cords at 48 hours after surgery. The mRNA levels of C-C motif chemokine 2 (CCL2), C-X-C motif chemokine ligand 1 (CXCL1), interleukin (IL)-1β, IL-6, tumor necrosis factor (TNF)-α, B-cell lymphoma 2 (Bcl-2), B-cell lymphoma-extra large (Bcl-XL), cystathionine beta synthase, cystathionine gamma lyase, 3-mercaptopyruvate sulfurtransferase, sulfide:quinone oxidoreductase (SQOR), ethylmaronic encephalopathy 1, thiosulfate sulfurtransferase, sulfite oxidase, cysteinyl-tRNA synthetase 1, and cysteinyl-tRNA synthetase 2 were standardized to the level of 18S ribosomal RNA using quantitative real-time polymerase chain reaction (7500 Fast Real-Time PCR System, Thermo Fisher Scientific, Waltham, MA, USA). Primer sequences are listed in Table 2.

TABLE 2
List of primer sequences for
quantitative polymerase chain reaction.

| | | | SEQ |
|---|---|---|---|
| | | | ID |
| Target | F/R | Sequence | NO: |
| IL-1β | Forward | 5′-TGTAATGAAAGACGGCACACC-3′ | 1. |
| | Reverse | 5′-TCTTCTTTGGGTATTGCTTGG-3′ | 2. |
| IL-6 | Forward | 5′-CCGGAGAGGAGACTTCACAGA-3′ | 3. |
| | Reverse | 5′-CAGAATTGCCATTGCACAAC-3′ | 4. |
| TNF-α | Forward | 5′-CAGCCTCTTCTCATTCCTGC-3′ | 5. |
| | Reverse | 5′-GGTCTGGGCCATAGAACTGA-3′ | 6. |
| Bcl-2 | Forward | 5′-AGCGTCAACAGGGAGATGTCAC-3′ | 7. |
| | Reverse | 5′-GGGCCATATAGTTCCACAAAGGC-3′ | 8. |
| CBS | Forward | 5′-CGGACTCCCCACATTATCAC-3′ | 9. |
| | Reverse | 5′-CACACTTGAGACCGGCATTC-3′ | 10. |
| CSE | Forward | 5′-CTTGCTGCCACCATTACGATT-3′ | 11. |
| | Reverse | 5′-TCTTCAGTCCAAATTCAGATGCC-3′ | 12. |
| 3-MST | Forward | 5′-TCACAGCCGCTGAAGTTACTG-3′ | 13. |
| | Reverse | 5′-CAGCATGTGGTCGTAGGGG-3′ | 14. |
| ETHE1 | Forward | 5′-CTGTCATCTCCCGCCTCA-3′ | 15. |
| | Reverse | 5′-GCTCGAGTCTCCAAAGCAA-3′ | 16. |
| TST | Forward | 5′-CCAGCTGGTGGACTCTCG-3′ | 17. |
| | Reverse | 5′-GTGGCCCGAGTCTAGTCCT-3′ | 18. |
| SQOR | Forward | 5′-CCCGGCTCTTTGCCTGTTT-3′ | 19. |
| | Reverse | 5′-CCAGCACCTCATAGTGGTTCTT-3′ | 20. |
| SUOX | Forward | 5′-TCTACCATGAGCATCGGTGT-3′ | 21. |
| | Reverse | 5′-CATCGAAGACCTCAGAGCCTA-3′ | 22. |
| 18S | Forward | 5′-CGGCTACCACATCCAAGGAA-3′ | 23. |
| | Reverse | 5′-GCTGGAATTACCGCGGCT-3′ | 24. |
| CCL2 | TaqMan | Mm99999056_m1 | |
| CXCL1 | TaqMan | Mm01354329_g1 | |
| Bcl-XL | TaqMan | Mm00437783_m1 | |
| CARS1 | TaqMan | Mm01133476_m1 | |
| CARS2 | TaqMan | Mm01209062_m1 | |
| 18S | TaqMan | Mm04277571_s1 | |

Abbreviations:
Bcl-2, B-cell lymphoma 2;
Bcl-XL, B-cell lymphoma-extra large;
CARS, cysteiny1-tRNA synthetase;
CBS, cystathionine beta synthase;
CCL2, C-C motif chemokine 2;
CSE, cystathionine gamma lyase;
CXCL1, C-X-C motif chemokine ligand 1;
ETHE1, ethylmaronic encephalopathy 1;
IL, interleukin;
SQOR, sulfide: quinone oxidoreductase;
SUOX, sulfite oxidase;
3-MST, 3-mercaptopyruvate sulfurtransferase;
TNF-α, tumor necrosis factor-α;
TST, thiosulfate sulfurtransferase.

### Liquid Chromatography-Tandem Mass Spectrometry (LC-MS/MS) Analysis

We used LC-MS/MS analysis to measure the amount of ³⁴S-labeled GSSSG ([³²S₂, ³⁴S]GSSSG) in the CNS and plasma after intranasal administration of GS³⁴SSG, an isotope of endogenous GSSSG^{9,35,36}. The ratios of ³⁴S-labeled sulfane sulfur species ([³²S, ³⁴S]GSSH, [³²S, ³⁴S]cysteine hydropersulfide (CysSSH), and [³²S₂, ³⁴S]cysteine trisulfide (CysSSSCys)) to endogenous sulfane sulfur species ([³²S₂]GSSH, [³²S₂]CysSSH, and [²S₃]CysSSSCys) were also quantitatively evaluated. At 30 minutes after intranasal administration of 50 mg/kg of GS³⁴SSG, blood was obtained and four different central nervous tissues were harvested: olfactory bulb and forebrain; brainstem; cervical and thoracic spinal cord, and lumbar spinal cord. A total of 30 mg of each tissue was homogenized in 300 μl of ice-cold methanol solution containing 5 mM β-(4-hydroxyphenyl)ethyl iodoacetamide (HPE-IAM; Santa Cruz Biotechnology, Dallas, TX, USA). Twenty-five μl of plasma was mixed with 75 μl of ice-cold methanol solution containing 5 mM HPE-IAM. Samples were incubated for 20 minutes at 37° C. in the dark. After centrifugation (14,000×g for tissues, 1,870×g for plasma, 10 minutes, 4° C.), the supernatant was separated and diluted with 0.1% formic acid for LC-MS/MS analysis, and the pellet was sonicated to measure the protein concentration by BCA assay. The amount of [³²S₂, ³⁴S]GSSSG was quantified in selective reaction monitoring (SRM) with precursor ion (647.14 m/z), product ion (389.1 m/z), and higher energy collisional dissociation (21 v). The ratios of [³²S, ³⁴S]GSSH to [³²S₂]GSSH, [³²S, ³⁴S]CysSSH to [³²S₂]CysSSH, and [³²S₂, ³⁴S]CysSSSCys to [³²S₃]CysSSSCys were calculated from their peak area measured by Dionex UltiMate 3000 RS UPLC-Orbitrap Exploris 480 mass spectrometer (Thermo Fisher Scientific, Waltham, MA, USA). In brief, samples were subjected to the UPLC system with a Hypersil Gold C-18 (100×2.1 mm, 3.0 μm, Thermo Fisher Scientific, Waltham, MA, USA) column and were then eluted by a linear methanol gradient of the mobile phase (0-90%, 15 minutes) in the presence of 0.1% formic acid at a flow rate of 0.2 ml/minute at 40° C. The raw data were analyzed by Compound Discoverer 3.3 software (Thermo Fisher Scientific, Waltham, MA, USA). The molecular weights of these sulfane sulfur species combined with HPE-IAM were determined based on previous reports^{9,11,37}.

Measurements of Relative Sulfane Sulfur Levels in Lumbar Spinal Cords after Spinal Cord Ischemia

Lumbar spinal cords were harvested 48 hours after surgery and snap-frozen in liquid nitrogen. Spinal cords were subsequently homogenized in Hanks' balanced salt solution (HBSS; Thermo Fisher Scientific, Waltham, MA, USA) containing SSip-1 (5 μM), incubated at room temperature in the dark for 20 minutes, and then subjected to centrifugation. SSip-1 was synthesized and provided by the Hanaoka laboratory³⁸. The fluorescence intensities of supernatants were measured using a microplate reader (SpectraMax M5; Molecular Devices, San Jose, CA, USA) at the wavelength of λₑₓ/λₑₘ=491 nm/525 nm. The fluorescence intensities were normalized to the weights of the spinal cord.

Measurements of Relative Sulfane Sulfur Levels in Olfactory Bulb and Forebrain, and Whole Spinal Cord after Intranasal Administration of PTN-SSS

At 30 minutes after intranasal administration of 50 mg/kg of PTN-SSS, the olfactory bulb and forebrain and the whole spinal cord were harvested. Tissues were homogenized in HBSS containing 10 M of SSip-1. The fluorescence intensities of supernatants were measured.

### In Vitro Studies

### Murine Primary Cortical Neuron Culture

Primary cortical neurons were isolated from the cerebral cortices of C57BL/6J mice of both sexes at embryonic day 15, as previously described 39. Primary cortical neurons were maintained at 37° C. in a humidified tissue culture chamber with 5% CO₂ and cells were used on day 11 after harvest.

Measurements of Relative Sulfane Sulfur Levels in Primary Cortical Neurons or SH-SY5Y Cells after Incubation with Polysulfides

Primary cortical neurons or SH-SY5Y cells, a human neuroblastoma cell line (American Type Culture Collection, Manassas, VA, USA), were incubated with SSip-1 DA (5 M)³⁸ at 37° C. for 20 minutes in the dark. Cells were washed with pre-warmed HBSS containing calcium and magnesium (Thermo Fisher Scientific, Waltham, MA, USA) and then cells were incubated with GSSSG, sodium trisulfide (Na₂S₃; Dojindo Molecular Technologies, Inc., Rockville, MD, USA), PTN-SSS, or vehicle alone for 20 minutes at 37° C. in the dark. Fluorescence intensities were measured using a microplate reader.

Effect of Polysulfides on Cell Viability after Oxygen and Glucose Deprivation/Reoxygenation (OGD/R)

After primary cortical neurons or SH-SY5Y cells were subjected to 2.5 or 15 hours of OGD in an incubation chamber (MIC-101; Billups-Rothenberg, Inc., San Diego, CA, USA), the cells were incubated with GSSSG, GSH, GSSG, PTN-SSS, or vehicle only, and then subjected to 21- (primary cortical neurons) or 24- (SH-SY5Y cells) hours of reoxygenation, as previously described³⁹. After reoxygenation, cell viability and cell injury were assessed using crystal violet and lactate dehydrogenase (LDH) assays, as previously described³⁹.

### Cytotoxic Effect of GSSSG on Cell Viability

Primary cortical neurons were incubated for 21 hours with GSSSG at 0 μM (vehicle alone), 10 μM, 30 μM, 60 μM, or 100 μM in a humidified incubator with 95% air and 5% CO₂ at 37° C. Cell viability was assessed using the LDH assay.

### Cytotoxic Effect of PTN-SSS on Cell Viability

SH-SY5Y cells were incubated for 24 hours with PTN-SSS at 0 μM (vehicle alone), 5 μM, 10 μM, 25 μM, 50 μM, or 100 μM in a humidified incubator with 95% air and 5% CO₂ at 37° C. Cell viability was assessed using the crystal violet assay.

### Statistical Analysis

Data are presented as means with standard deviation (parametric data) or medians with interquartile range (nonparametric data). Descriptive statistics were used to describe the study population. We used Shapiro-Wilk test and Q-Q plots to assess the normality of the data. Parametric data were analyzed using unpaired t test to compare two groups, and one-way analysis of variance (ANOVA) with Tukey's multiple comparison test or Dunnett's multiple comparison test to compare three or more groups. Non-parametric data were analyzed using Mann-Whitney test to compare two groups, and Kruskal-Wallis test with Dunn's multiple comparisons test to compare three or more groups. The significance was considered at the level of P<0.05. Statistical analyses were conducted using GraphPad Prism 9.2.0 (GraphPad Software Inc., San Diego, CA, USA).

The sample size calculation of the experiment to compare the BMS scores at 72 hours after surgery between GSSSG, GSH, and vehicle alone groups was conducted using F test for the fixed effects one-way ANOVA (G*Power 3.1; Heinrich-Heine-Universitat, Dusseldorf, Germany)⁴⁰. We presumed that 11 mice for male mice and 8 mice for female mice per group would be required based on our pilot studies for this experiment (α=0.05, β=0.1 [Power=0.9], effect size f=0.7022756 for male mice and f=0.8709832 for female mice, number of groups=3). The sample size calculation of the experiment to compare the BMS scores at 72 hours after surgery between PTN-SSS, PTN, and vehicle alone groups was conducted using F test for the fixed effects one-way ANOVA. We presumed that 9 mice per group would be required based on our pilot studies for this experiment (α=0.05, β=0.1 [Power=0.9], effect size f=0.7747206, number of groups=3).

Intermittent H₂S Inhalation—Sulfide Pre-Conditioning (SPC) We previously reported that breathing H₂S at 80 ppm markedly increases plasma and brain levels of H₂S and thiosulfate, an oxidative product of H₂S.^{50,51} Male C57BL6/J mice breathed air alone and air mixed with 80 ppm H₂S (Airgas Inc., Radnor, PA) for 4 h each day for 5 consecutive days in custom-made plastic chambers. The H₂S breathing session was performed from 8 AM until 12 PM from Day 1 through Day 5 as previously described⁵¹ (FIG. 1). H₂S concentration, as well as FiO₂, was continuously measured using a portable gas monitor (VENTIS MX4 Multi-Gas Monitor, Industrial Scientific Corporation, Oakdale. PA).

### MPTP-Induced Parkinson's Disease Model in Mice

Male C57BL6/J and CD-1 mice received four intraperitoneal injections of MPTP (20 mg/kg) or saline every 2 h for a total of four doses of MPTP (total, 80 mg/kg) or equivalent amount of saline, as previously described 3. To evaluate the effect of MPTP on dopaminergic neurons, all mice were harvested 7 days after MPTP administration. Then, tyrosine hydroxylase protein levels were detected by immunoblotting.

### Cell Viability of Neuronal Cells

Cell viability assays was performed to examine cell viabilities of SH-SY5Y cells or murine primary cortical neurons 24 h after the addition of MPP⁺ (5 mM for SH-SY5Y, 50 μM for primary cortical neurons) with vehicle (PBS), Na₂S (Sigma-Aldrich), Na₂S₃ (Dojindo Molecular Technologies, Inc), GSSSG (Glutathione trisulfide, Kyowa Hakko Bio Co., Ltd., Tokyo, Japan), LA (α-Lipoic acid, Sigma-Aldrich) or LASSS (α-Lipoic acid trisulfide, Kyowa Hakko Bio Co., Ltd., Tokyo, Japan). Cell viability was measured using the crystal violet assay.

Administration of Na₂S₃ to Mice after Treatment with MPTP

To determine the effects of polysulfide on the loss of tyrosine hydroxylase in the substantia nigra and striatum in mice treated with MPTP, we administered Na₂S₃ (20 mg/kg) or saline every 2 h for intraperitoneally, starting immediately after the first injection of MPTP on Day 0. Subsequently, mice received two intraperitoneal injections of Na₂S₃ (20 mg/kg) or saline every 12 h from day 1 to day 6. All mice were harvested 7 days after MPTP administration. Then, tyrosine hydroxylase protein levels were detected by immunoblotting.

Administration of GSSSG to Mice after Treatment with MPTP

To determine the effects of polysulfide on the loss of tyrosine hydroxylase in the substantia nigra and striatum in mice treated with MPTP, we administered GSSSG (50 mg/kg) or saline via intranasal route immediately after the first and the third injection of MPTP on Day 0. Subsequently, mice received two intranasal injections of GSSSG(50 mg/kg) or saline every 12 h from day 1 to day 6. All mice were harvested 7 days after MPTP administration. Then, tyrosine hydroxylase protein levels were detected by immunoblotting.

### Example 1. Intranasal Administration of GSSSG Rescued Mice from Delayed Paraplegia after Transient Spinal Cord Ischemia

To investigate whether polysulfides can prevent delayed paraplegia, mice were subjected to SCI and were treated with GSSSG, GSH, or vehicle alone 0, 8, 23, and 32 hours after surgery. Thirty-six male mice were subjected to SCI; however, three of these mice were excluded from further study because a greater than 90% reduction in the femoral artery blood flow was not achieved. The remaining 33 male mice received one of three treatments: GSSSG (n=11), GSH (n=11), or vehicle alone (n=11). The hindlimb motor function was quantified using the Basso mouse scale for locomotion (BMS), which evaluates hindlimb movement, forelimb-hindlimb coordination, and trunk stability. As previously described by Kakinohana and colleagues, if the neurologic deficit (BMS<6) occurred after a period during which a mouse was able to walk (BMS≥6), then the mouse was considered to have delayed paraplegia⁶.

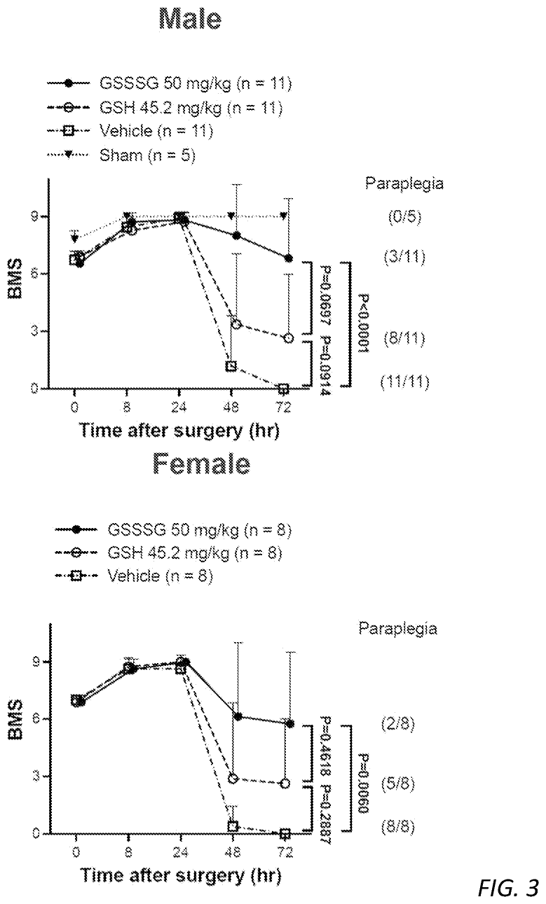

Beginning approximately 36 hours after surgery, the hindlimb motor function of mice treated with vehicle alone gradually worsened and all of the mice treated with vehicle alone developed paraplegia by 48 hours after surgery (FIG. 3). In contrast, intranasal administration of GSSSG prevented the development of delayed paraplegia in 8 of 11 mice (73%) and, compared to vehicle alone, improved the BMS score at 72 hours after surgery (GSSSG vs vehicle alone, BMS; 9 [4-9] vs 0 [0-0]; P<0.0001 by Kruskal-Wallis test with Dunn's multiple comparisons test, FIG. 3). Intranasal administration of GSH also prevented delayed paraplegia in 3 of 11 mice (27%), but did not improve the BMS score at 72 hours after surgery (GSH vs vehicle alone, BMS; 1 [0-7] vs 0 [0-0]; P=0.0914, FIG. 3). The results show that GSSSG, but not GSH, prevents the development of delayed paraplegia after SCI in male mice.

To investigate the effect of sex on the ability of GSSSG to prevent delayed paraplegia after SCI, 28 female mice were subjected to SCI and were treated with GSSSG, GSH, or vehicle alone 0, 8, 23, and 32 hours after surgery. Four of the 28 female mice experienced labored breathing immediately after extubation and were euthanized. The remaining 24 female mice were treated with GSSSG (n=8), GSH (n=8), or vehicle alone (n=8). Compared to mice treated with vehicle alone, mice that received intranasal GSSSG had an improved BMS scores at 72 hours after surgery (GSSSG vs vehicle alone, BMS; 6.5 [1.5-9.0] vs 0.0 [0.0-0.0]; P=0.0060 by Kruskal-Wallis test with Dunn's multiple comparisons test, FIG. 3). In contrast, intranasal administration of GSH did not improve the BMS scores at 72 hours after surgery (GSH vs vehicle alone, BMS; 0.5 [0.0-6.7] vs 0.0 [0.0-0.0]; P=0.2887, FIG. 3). These results show that GSSSG, but not GSH, is able to prevent delayed paraplegia after SCI in both sexes.

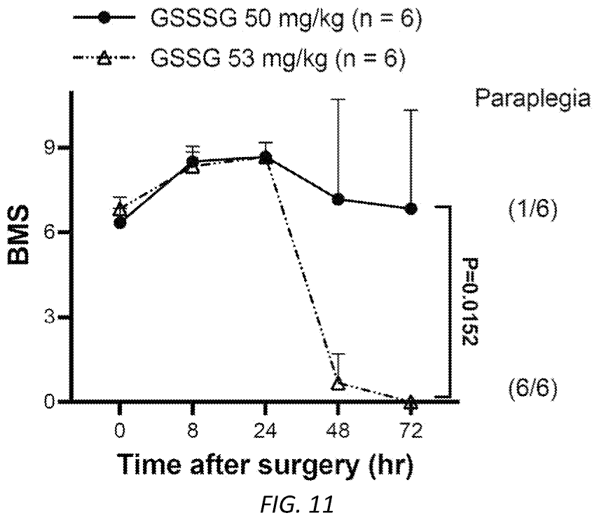

GSSG can react with thiols in proteins by glutathionylation (PSH+GSSG⇄PSSG+GSH)²³. GSSSG potentially can also react with thiols in proteins by the same process⁴¹. To investigate whether the beneficial effects of GSSSG are related to glutathionylation, we compared the effects of intranasal administration of GSSSG or GSSG on outcomes after SCI in male mice. Intranasal administration of GSSSG rescued 5 out of 6 male mice (83%) from delayed paraplegia; in contrast, intranasal administration of GSSG did not rescue any male mouse from delayed paraplegia (0%). The BMS score at 72 hours after surgery in the GSSSG group was significantly higher than that in the GSSG group (GSSSG vs GSSG, BMS; 8.0 [5.2-9.0] vs 0.0 [0.0-0.0]; P=0.0152 by Mann Whitney test, FIG. 11). Because GSSSG potentially can mediate glutathionylation of target proteins by the same process as GSSG, but only GSSSG protects against delayed paraplegia, the results suggest that the neuroprotective effects of GSSSG are not a result of glutathionylation.

### Example 2. Intranasal Administration of GSSSG Decreased Neurodegeneration and Microglial- and Caspase-3-Activation after Spinal Cord Ischemia in Lumbar Spinal Cords

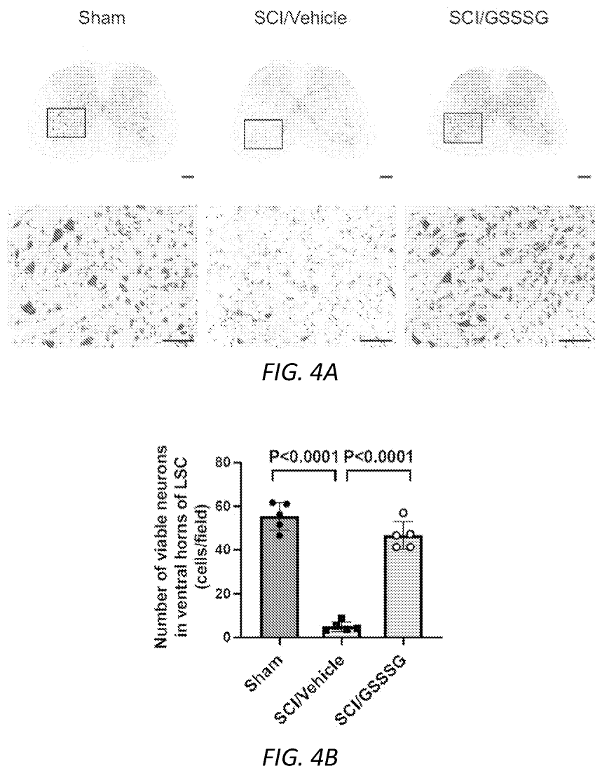

Spinal cord ischemia is associated with degenerative changes in neurons in the ventral horn of the lumbar spinal cord and the viability of affected neurons can be assessed using Nissl staining⁶. Nissl stain detects Nissl bodies in the cytoplasm of neurons and the presence of this purple, cytoplasmic staining is an indicator of neuronal integrity^{6,42}. To investigate the effects of GSSSG on neurodegeneration, GSSSG or vehicle alone was administered 0, 8, 23, and 32 hours after surgery. Lumbar spinal cords were harvested 48 hours after surgery, fixed, sectioned, and incubated with Nissl stain. Compared to mice undergoing a sham surgical procedure, spinal cord ischemia was associated with a marked decrease in neurons that were positive for Nissl stain, suggesting that the majority of cells were not viable (FIGS. 4A-B). Compared to mice that underwent SCI and were treated with vehicle alone, intranasal administration of GSSSG was associated with a significant increase in the number of viable neurons (FIGS. 4A-B).

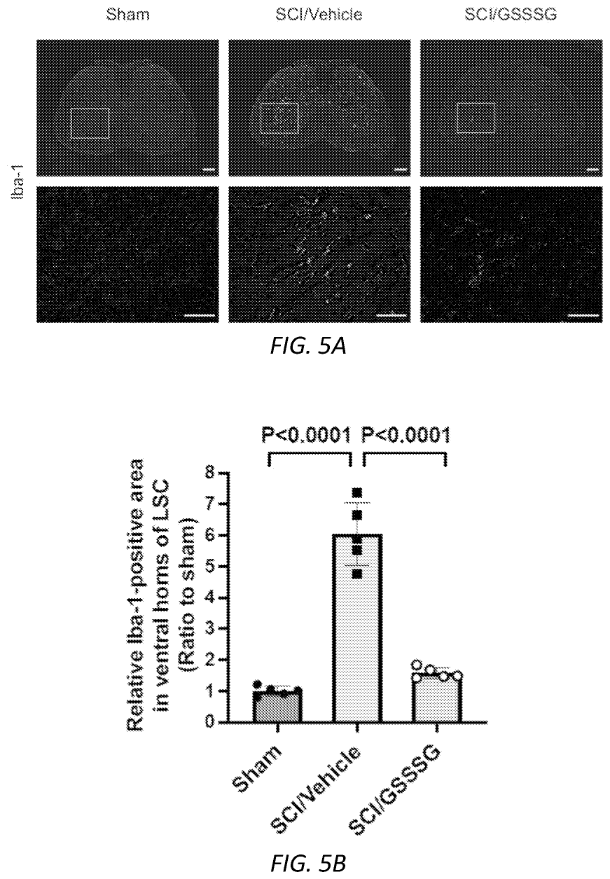

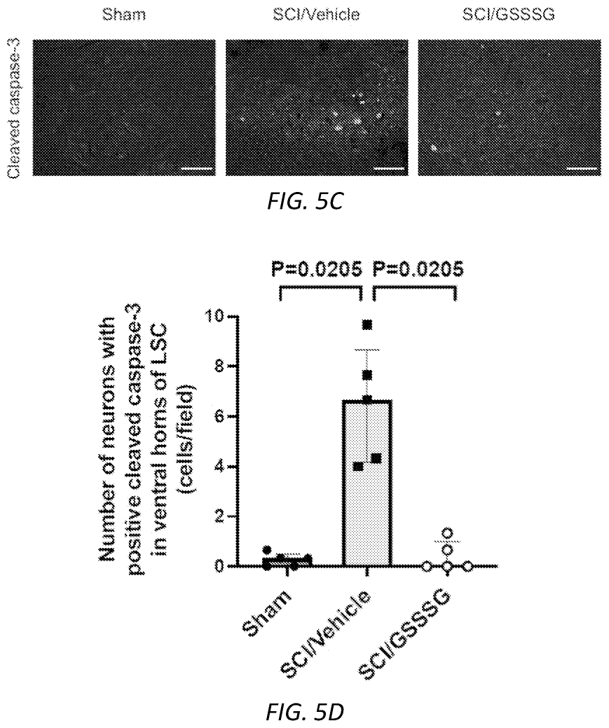

Previous studies showed that increased microglial activation in the ventral horn of the lumbar spinal cord parallels the onset of delayed paraplegia after SCI^{6,29}, and inhibition of microglial activation attenuates neuronal injury and prevents the development of delayed paraplegia after SCI⁴³. Increased expression of Ionized calcium-binding adapter molecule 1 (Iba-1) is a sensitive marker for activation of microglia⁴⁴. To explore the mechanisms by which GSSSG prevents neurodegeneration, mice were subjected to SCI and treated with GSSSG or vehicle alone. Sections were prepared from the lumbar spinal cord and were stained for Iba-1. Compared to the staining of the spinal cord obtained from mice 48 hours after a sham operation, the staining of the spinal cord from mice that were subjected to SCI had a markedly increased relative area of Iba-1-positive staining (FIGS. 5A, 5B). The results indicate that SCI is associated with increased activation of microglia. In contrast, mice that underwent SCI and were treated with intranasal GSSSG had a significant reduction in staining for Iba-1, indicating decreased microglial activation (FIGS. 5A, 5B).

Previous investigators showed that activation of caspase-3 is a central component of the neurodegeneration that occurs in the ventral horn of the lumbar spinal cord after SCI⁶. Immunohistochemical staining for cleaved caspase-3 was used to investigate whether attenuation of caspase-3 activation may be a mechanism by which GSSSG prevents neurodegeneration after SCI. Compared to spinal cords from mice 48 hours after a sham operation, the lumbar spinal cords from mice that were subjected to SCI had an increased number of cleaved caspase-3-positive neurons in the ventral horns (FIGS. 5C, 5D). Intranasal administration of GSSSG prevented the SCI-induced increase in the number of cleaved caspase-3-positive neurons in lumbar spinal cord (FIGS. 5C, 5D). These results suggest that the intranasal administration of GSSSG prevents neurodegeneration in the ventral horn of the lumbar spinal cord and is associated with decreased microglial activation and attenuation of caspase-3 activation.

### Example 3. Intranasal Administration of GSSSG Attenuated Upregulation of Pro-Inflammatory Cytokines after Spinal Cord Ischemia

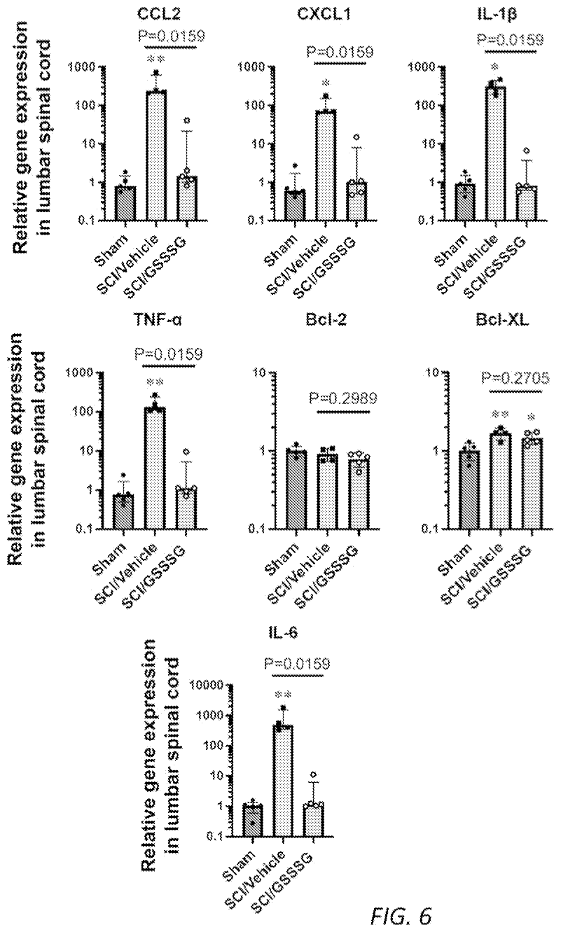

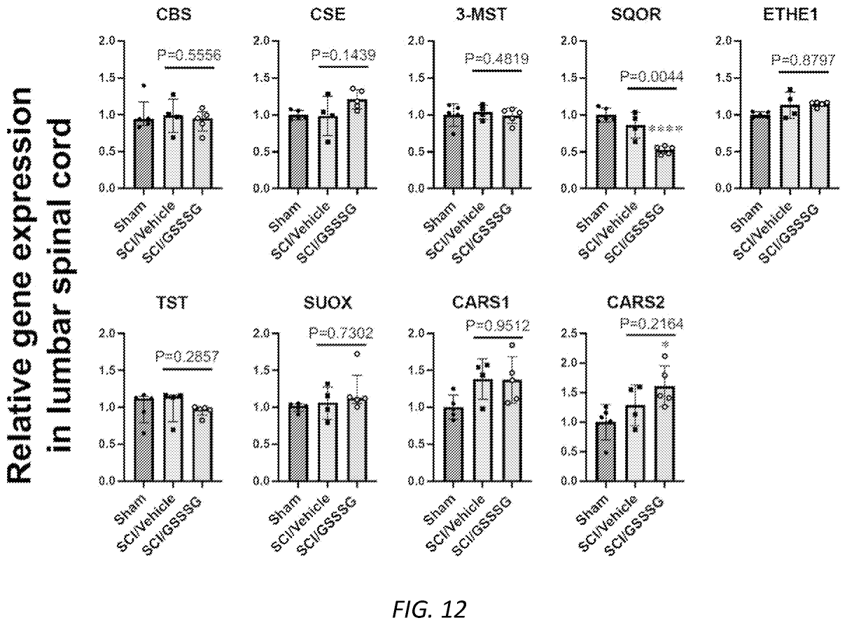

Previous studies showed that a marked increase in pro-inflammatory cytokines produced by activated microglia precedes the onset of delayed paraplegia after SCI^{15,29}. Inhibition of microglial activation suppresses the upregulation of pro-inflammatory cytokines and prevents the development of delayed paraplegia after SCI⁴³. To further assess the effect of GSSSG on pro-inflammatory cytokines induced by SCI, we measured the levels of mRNA encoding CCL2, CXCL1, IL-10, IL-6, and TNF-α in lumbar spinal cords from mice that were subjected to SCI and treated with GSSSG or vehicle alone. Lumbar spinal cords were harvested 48 hours after surgery. Compared to mice that underwent a sham operation, mice that were subjected to SCI had a marked increase in the levels of mRNAs encoding cytokines associated with inflammation. In contrast, GSSSG attenuated the upregulation of mRNA encoding pro-inflammatory cytokines 48 hours after surgery (FIG. 6). The intranasal administration of GSSSG also decreased the level of mRNA encoding SQOR, an enzyme that oxidizes sulfides to persulfides. GSSSG had no effect on mRNA levels encoding other enzymes that synthesize or metabolize sulfides or persulfides (FIG. 12). There was no significant difference between vehicle alone and GSSSG treatment groups in mRNA levels of anti-apoptotic genes including Bcl-2 and Bcl-XL (FIG. 6). These results suggest that the beneficial effects of GSSSG may be mediated by inhibition of pro-inflammatory cytokines.

### Example 4. ³⁴S-Labeled GSSSG Reached the Spinal Cord Shortly after Intranasal Administration and was Metabolized to Other Sulfane Sulfur Species

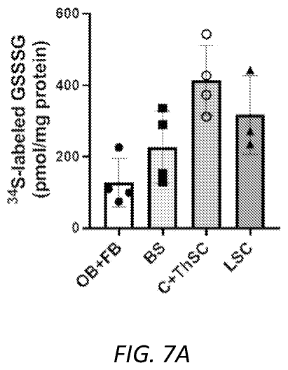

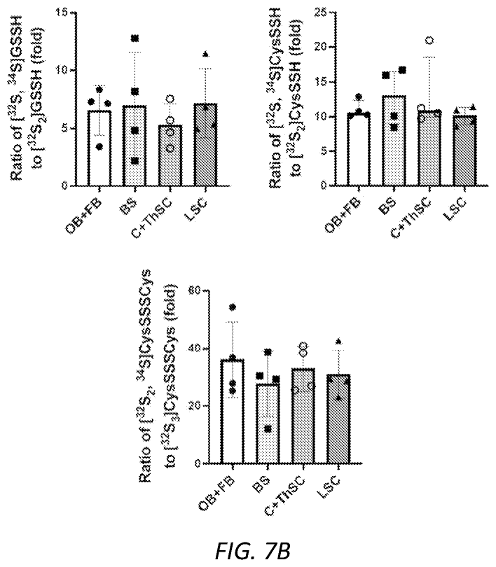

To study the pharmacokinetics of intranasally-administered GSSSG, we used ³⁴S-labeled GSSSG to investigate the distribution of GSSSG and its metabolites (GSSH, CysSSH, and CysSSSCys) in the CNS after intranasal administration. Thirty minutes after intranasal administration of GS³⁴SSG, LC-MS/MS was used to measure the amount of [³²S₂, ³⁴S]GSSSG, and to determine the ratios of ³⁴S-labeled sulfane sulfur species to endogenous sulfane sulfur species, in brain, spinal cord, and plasma. [³²S₂, ³⁴S]GSSSG was detected in olfactory bulb and forebrain (128±67 pmol/mg protein), brainstem (226±101 pmol/mg protein), cervical and thoracic spinal cord (414±98 pmol/mg protein), and lumbar spinal cord (317±111 pmol/mg protein) (FIG. 7A). In addition, [³²S₂, ³⁴S]GSSSG was detected in plasma at a concentration of 25±10 nM. The ratios of [³²S, ³⁴S]GSSH to [³²S₂]GSSH, [³²S, ³⁴S]CysSSH to [³²S₂]CysSSH, and [³²S₂, ³⁴S]CysSSSCys to [³²S₃]CysSSSCys in brain and spinal cord were 6.5±2.8, 10.6 [9.8-12.5], and 31.8±9.8 respectively (FIG. 7B). These results show that GSSSG reaches different parts of the central nervous system, including the spinal cord, within 30 minutes after intranasal administration, and is converted to other sulfane sulfur species.

### Example 5. Intranasal Administration of GSSSG Increased Sulfane Sulfur Levels in Lumbar Spinal Cords after Spinal Cord Ischemia

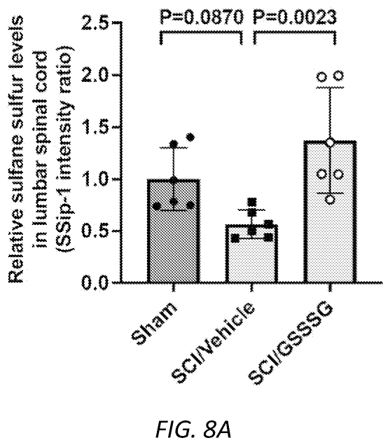

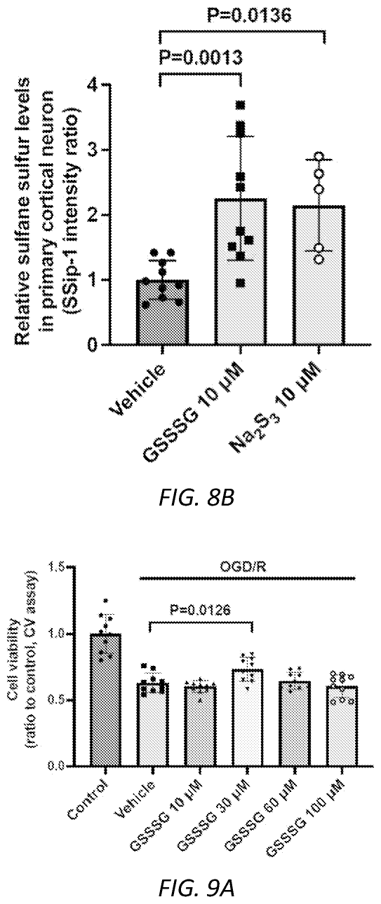

GSSSG protects the lumbar spinal cord from neurodegeneration 48 hours after SCI. To investigate whether increased levels of sulfane sulfur in the lumbar spinal cord might contribute to the neuroprotective effect, we measured the change in sulfane sulfur levels 48 hours after SCI. Compared to mice that underwent a sham operation, SCI followed by treatment with vehicle alone did not alter sulfane sulfur levels in the lumbar spinal cords (sham operation vs SCI followed by vehicle alone; 1.00±0.30 vs 0.56±0.13; P=0.0870 by one-way ANOVA with Dunnett's multiple comparison test, FIG. 8A). In contrast, the levels of sulfane sulfur in the lumbar spinal cord in mice that underwent SCI and received intranasal administration of GSSSG were greater than those in mice that underwent SCI and received vehicle alone (SCI followed by GSSSG vs vehicle alone; 1.37±0.50 vs 0.56±0.13; P=0.0023, FIG. 8A). These results suggest that the beneficial effects of GSSSG in preventing delayed paraplegia are associated with increased sulfane sulfur levels in lumbar spinal cords.

### Example 7. Effects of GSSSG on Primary Cortical Neurons

To determine whether polysulfides increase sulfane sulfur levels within neurons, we measured relative sulfane sulfur levels in primary cortical neurons using SSip-1 DA, in the presence and absence of polysulfides. SSip-1 DA is a fluorescent probe that can be used to measure the concentration of sulfane sulfur inside cells³⁸. Relative to untreated primary cortical neurons, neurons that were incubated with GSSSG or Na₂S₃ had increased levels of intracellular sulfane sulfur (FIG. 8B).

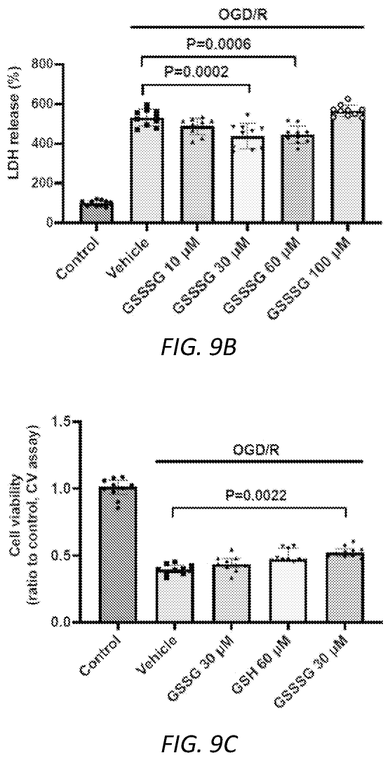

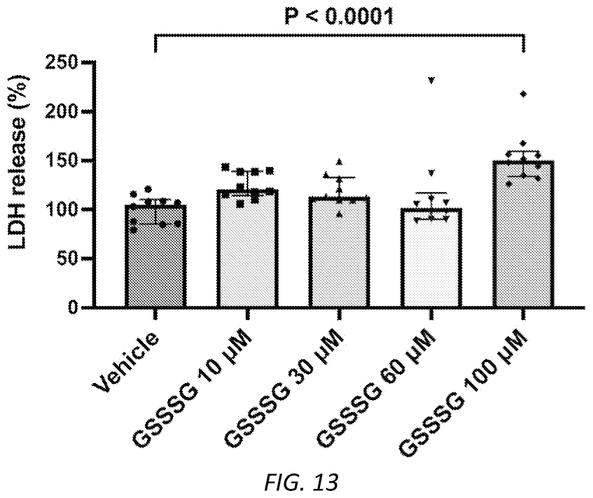

In vivo studies showed that GSSSG can prevent neurodegeneration after SCI. To determine whether GSSSG can protect neurons from similar injury in vitro, primary cells were incubated with GSSSG after oxygen and glucose deprivation/reoxygenation (OGD/R). Cell viability was assessed using crystal violet and lactate dehydrogenase (LDH) assays, as previously described 39. Crystal violet dye stains DNA and protein in cells and confirms that the cells are alive and able to maintain attachment to tissue culture plates despite OGD/R. The LDH assay measures the level of lactate dehydrogenase in tissue culture medium and is an indirect measure of plasma membrane damage. Compared to control cells, treatment with OGD/R was associated with a marked decrease in the number of live, adherent cells (FIG. 9A) and with a marked increase in the concentration of LDH in the tissue culture medium (FIG. 9B). Compared to vehicle-only treated cells, GSSSG at 30 μM increased cell viability as measured by the crystal violet assay (FIG. 9A), and GSSSG at 30 μM or 60 μM decreased release of LDH after OGD/R (FIG. 9B). A higher dose of GSSSG (100 μM) per se increased LDH levels in the tissue culture medium (FIG. 13) and failed to improve cell survival after OGD/R. These results show that GSSSG dose-dependently protects neurons from the effects of OGD/R.

In vivo studies showed that GSSSG, but not GSH or GSSG, prevents the development of delayed paraplegia after SCI, and suggested that the neuroprotective effects of GSSSG are derived from sulfane sulfur. To determine whether the neuroprotective effects of GSSSG on primary cultured neurons are derived from sulfane sulfur, we tested the ability of GSSSG, GSH, and GSSG to protect the neurons from OGD/R. Compared to vehicle-only treated cells, GSSSG at 30 μM improved cell viability after OGD/R (FIG. 9C). In contrast, GSH at 60 μM and GSSG at 30 μM did not improve cell viability after OGD/R (FIG. 9C). These results suggest that the neuroprotective effects of GSSSG are derived from sulfane sulfur.

### Example 8. Effects of PTN-SSS on SH-SY5Y Cells

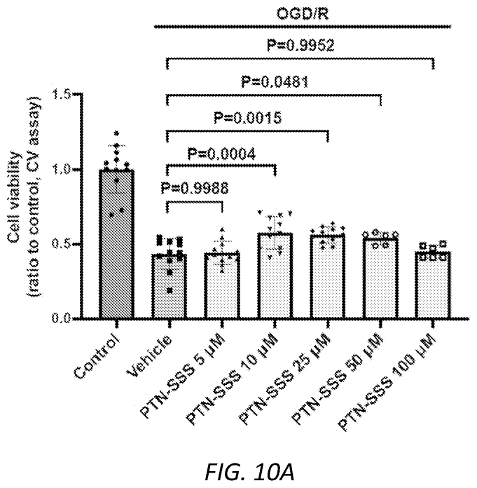

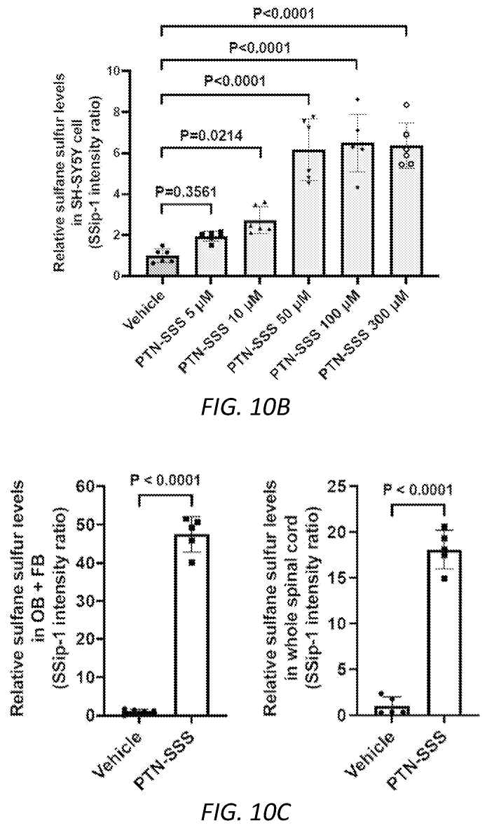

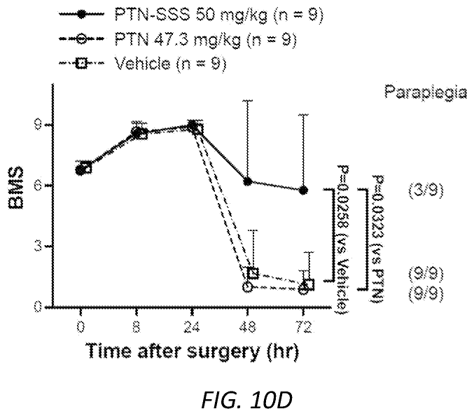

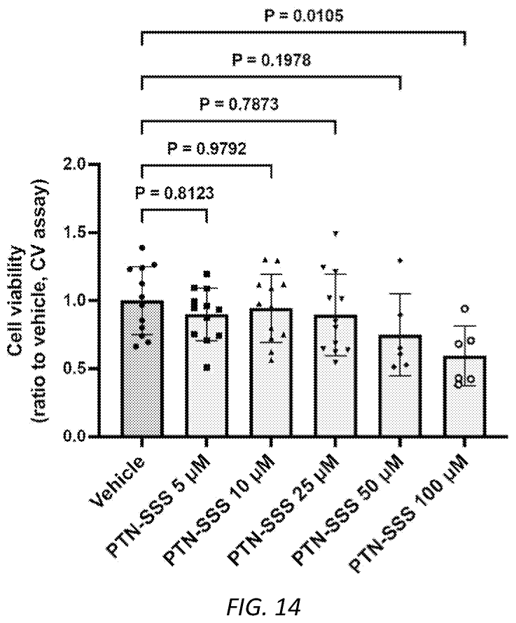

We used in vitro studies to show that GSSSG protects neurons after OGD/R, and to suggest that the neuroprotective effects of GSSSG are derived from sulfane sulfur. To investigate whether other polysulfides are cytoprotective after OGD/R, we examined the effect of PTN-SSS, another polysulfide, on the viability of SH-SY5Y cells after OGD/R. As with primary cortical neurons, treatment with OGD/R reduced the viability of SH-SY5Y cells. Compared to vehicle-only treated cells, incubation with PTN-SSS at 10 μM, 25 μM, or 50 μM significantly improved SH-SY5Y viability after OGD/R in a dose-dependent fashion (FIG. 10A). In contrast, PTN-SSS did not improve cell viability at 100 μM because of a direct cytotoxic effect of PTN-SSS at high doses (FIG. 14). These results show that PTN-SSS dose-dependently rescues SH-SY5Y cells after OGD/R, and support that polysulfides are cytoprotective after OGD/R.

To determine whether the cytoprotective effects of PTN-SSS are relative to the amount of intracellular sulfane sulfur, we used SSip-1 DA to measure sulfane sulfur levels in SH-SY5Y cells after incubation with PTN-SSS. The level of sulfane sulfur inside SH-SY5Y cells increased with increasing of concentration of PTN-SSS (FIG. 10B). The ability of SSip-1 DA (5 μM) to measure intracellular sulfane sulfur reached a maximum with the concentration of PTN-SSS at 100 μM (FIG. 10B). Compared to vehicle-only treated cells, the sulfane sulfur levels in SH-SY5Y cells increased significantly at the dose of PTN-SSS (10-50 μM), which significantly improved cell viability after OGD/R. These results suggest that the cytoprotective effects of PTN-SSS are relative to the amount of sulfane sulfur released from PTN-SSS.

### Example 10. PTN-SSS Increased the Sulfane Sulfur Levels in the Olfactory Bulb and Forebrain, and Whole Spinal Cord Shortly after Intranasal Administration

To study whether PTN-SSS increases sulfane sulfur levels in the central nervous system shortly after intranasal administration, we measured sulfane sulfur levels in the olfactory bulb and forebrain and the whole spinal cord 30 minutes after intranasal administration of PTN-SSS using SSip-1. Compared to mice that received intranasal administration of vehicle alone, intranasal administration of PTN-SSS increased sulfane sulfur levels in the olfactory bulb and forebrain and the whole spinal cord (FIG. 10C). These results suggest that PTN-SSS increases sulfane sulfur levels in the central nervous system within 30 minutes after intranasal administration.

### Example 11. Intranasal Administration of PTN-SSS Rescued Mice from Delayed Paraplegia after Transient Spinal Cord Ischemia

In vitro studies showed that PTN-SSS is cytoprotective after OGD/R, and the cytoprotective effects of PTN-SSS are relative to the amount of sulfane sulfur released from PTN-SSS. As GSSSG prevented the development of delayed paraplegia after SCI, and the neuroprotective effects of GSSSG were associated with sulfane sulfur, we hypothesized that PTN-SSS could also rescue mice from delayed paraplegia after SCI. In addition, as with GSSSG, we tested whether the sulfane sulfur in PTN-SSS, rather than PTN alone, was neuroprotective. Male mice received intranasal administration of PTN-SSS, PTN, or vehicle alone at 0, 8, 23, and 32 hours after surgery. An equimolar dose of PTN-SSS (50 mg/kg) or PTN (47.3 mg/kg) was administered. Intranasal administration of PTN-SSS rescued 6 of 9 male mice (66%) from delayed paraplegia. In contrast, intranasal administration of PTN did not rescue any male mouse from delayed paraplegia. The BMS score at 72 hours after surgery in the PTN-SSS group was significantly higher than that in the PTN or vehicle alone group (PTN-SSS vs PTN, BMS; 7.0 [1.5-9.0] vs 1.0 [0.0-2.0]; P=0.0323. PTN-SSS vs vehicle alone, BMS; 7.0 [1.5-9.0] vs 1.0 [0.0-1.5]; P=0.0258, FIG. 10D). These results show that PTN-SSS, but not PTN, prevents the development of delayed paraplegia after SCI in mice, indicating that the neuroprotective effects of PTN-SSS are derived from sulfane sulfur, not PTN.

### Example 12. Inhalation of H₂S Provided Neuroprotection in PD Animal Model

Sulfide levels are tightly controlled in the brain. We recently reported robust protective effects of sulfide catabolism in hypoxic/ischemic brain injury. See Marutani et al, Nature Communications 12, Article number: 3108; 25 May 2021. Because other forms of brain injury may be caused by altered sulfide homeostasis, we examined effects of sulfide catabolism in Parkinson's disease (PD), which is one of the most common neurodegenerative disorders. See also, Kida, K., Ichinose, F. (2015). Hydrogen Sulfide and Neuroinflammation. In: Moore, P., Whiteman, M. (eds) Chemistry, Biochemistry and Pharmacology of Hydrogen Sulfide. Handbook of Experimental Pharmacology, vol 230. Springer, Chain; Kida et al., Antioxidants & Redox Signaling 2010; 15:343-352; and Kida et al, Antioxidents and Redox Signaling 15(2): 343-52 (2011).

Administration of 1-methyl-4-phenyl-1,2,3,6-tetrahydropyridine (MPTP) caused loss of dopaminergic neurons, measured as levels of tyrosine hydroxylase (TH) in substantia nigra, and movement disorder in mice. Breathing air mixed with 80 ppm H₂S for 4 h each day for 5 consecutive days before administration of MPTP (sulfide preconditioning) protected mice from loss of dopaminergic neurons and movement disorder induced by MPTP. Protective effects of sulfide pre-conditioning were associated with marked increase in sulfide:quinone oxidoreductase (SQOR) levels in substantia nigra and striatum. AAV-mediated SQOR expression in the nigrostriatal region prevented MPTP-induced loss of dopaminergic neurons in mice.

This study revealed neuroprotective effects of sulfide catabolism by SQOR in a MPTP-induced mouse model of PD. Protective effects of upregulation of SQOR may be mediated by increasing persulfide levels.

### Example 13. Inhalation of H₂S Provided Neuroprotection in PD Animal Model

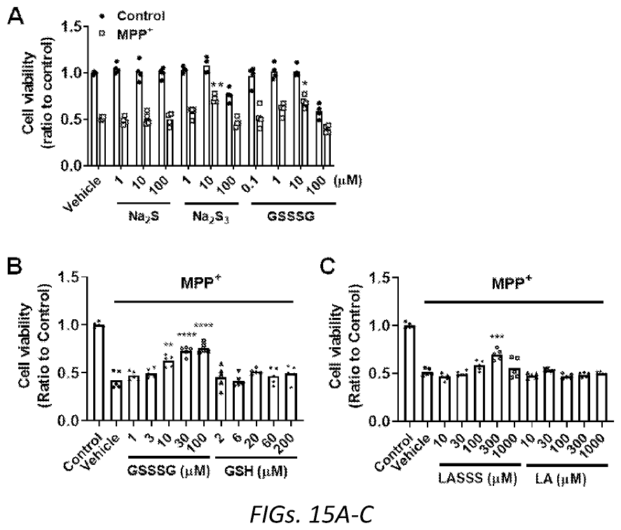

Because SQOR catalyzes the conversion of sulfide to polysulfides, and because SPC or AAV-mediated SQOR overexpression prevented the decrease in sulfane sulfur after MPTP administration, we hypothesized that the beneficial effects of upregulated SQOR after MPTP administration are mediated by increased levels of polysulfides. To consider this possibility, we examined the effects of polysulfides on the viability of SH-SY5Y cells incubated with MPP⁺. Cells were treated with MPP⁺ (5 mM) for 24 h and viability was assessed by crystal violet assay⁴¹. Compared to control cells, treatment with Na₂S (doses between 1 and 100 μM) did not improve the viability of SH-SY5Y cells treated with MPP^{+ 42}. In contrast, treatment with Na₂S₃ or GSSSG (10 μM each) increased the viability of MPP⁺-treated SH-SY5Y cells (FIG. 15A).

To further examine the cytoprotective effects of polysulfides in a physiologically relevant context, we examined the effect of GSSSG, glutathione (GSH, a parent molecule of GSSSG without a sulfane sulfur), α-lipoic acid trisulfide (1,2,3-trithiane-4-pentanoic acid, LA-SSS), and α-lipoic acid (a parent molecule of LA-SSS without a sulfane sulfur) on murine primary cortical neurons incubated with MPP⁺. Cells were incubated with MPP⁺ (50 μM) together with each of the above compounds for 24 hours. The polysulfide compounds GSSSG and LA-SSS, but not the parent compounds GSH and LA, protected murine primary conical neurons from the effects of MPP⁺ treatment (FIGS. 15B and C). These results suggest that the sulfane sulfur-releasing molecules protect neurons from MPP⁺-induced cytotoxicity.

### Example 14. Nasal Administration of GSSG Provided Neuroprotection in a PD Animal Model

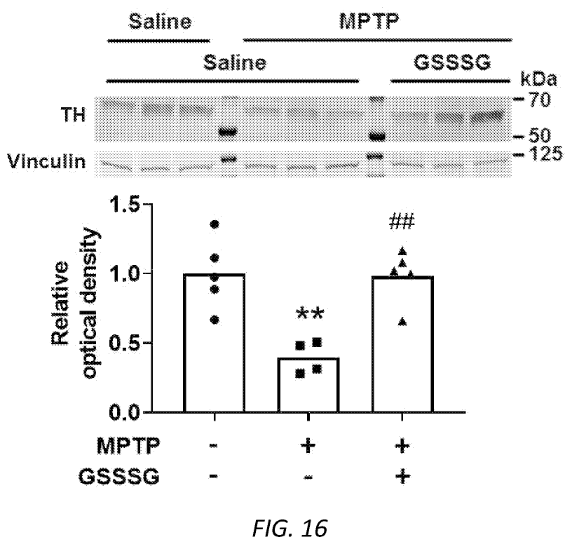

The large molecular weight of GSSSG (Mw: 644.7 DA) makes it unable to traverse the blood brain barrier after systemic administration. However, recent studies suggest that intranasal administration may permit successful passage of relatively large molecules into the central nervous system 43. To consider the possibility that GSSSG might have a protective effect on MPTP-induced neurodegeneration when administered intranasally, GSSSG (50 mg/kg) or saline was administered IN immediately after administration of MPTP or saline (control) on day 0. Between days 1 and 6, mice received GSSSG at 50 mg/kg or saline IN every 12 hours. We observed that administration of GSSSG IN prevented the MPTP-induced decrease in tyrosine hydroxylase in the nigrostriatal region (FIG. 16). Taken together, these results suggest that polysulfides have a robust therapeutic effect in a mouse model of PD.

### REFERENCES

1. Ziganshin B A, Elefteriades J A: Surgical management of thoracoabdominal aneurysms. Heart 2014; 100: 1577-82
2. Riambau V, Bockler D, Brunkwall J, Cao P, Chiesa R, Coppi G, Czerny M, Fraedrich G, Haulon S, Jacobs M J, Lachat M L, Moll F L, Setacci C, Taylor P R, Thompson M, Trimarchi S, Verhagen H J, Verhoeven E L, Esvs Guidelines C, Kolh P, de Borst G J, Chakfé N, Debus E S, Hinchliffe R J, Kakkos S, Koncar I, Lindholt J S, Vega de Ceniga M, Vermassen F, Verzini F, Document R, Kolh P, Black J H, 3rd, Busund R, Björck M, Dake M, Dick F, Eggebrecht H, Evangelista A, Grabenwöger M, Milner R, Naylor A R, Ricco J B, Rousseau H, Schmidli J: Editor's Choice—Management of Descending Thoracic Aorta Diseases: Clinical Practice Guidelines of the European Society for Vascular Surgery (ESVS). Eur J Vase Endovasc Surg 2017; 53: 4-52
3. Etz C D, Luehr M, Kari F A, Bodian C A, Smego D, Plestis K A, Griepp R B: Paraplegia after extensive thoracic and thoracoabdominal aortic aneurysm repair: does critical spinal cord ischemia occur postoperatively? J Thorac Cardiovasc Surg 2008: 135: 324-30
4. Ullery B W, Cheung A T, Fairman R M, Jackson B M, Woo E Y, Bavaria J, Pochettino A, Wang G J: Risk factors, outcomes, and clinical manifestations of spinal cord ischemia following thoracic endovascular aortic repair. J Vasc Surg 2011; 54: 677-84
5. Wang J F, Li Y, Song J N, Pang H G: Role of hydrogen sulfide in secondary neuronal injury. Neurochem Int 2014; 64: 37-47
6. Kakinohana M, Kida K, Minamishima S, Atochin D N, Huang P L, Kaneki M, Ichinose F: Delayed paraplegia after spinal cord ischemic injury requires caspase-3 activation in mice. Stroke 2011; 42: 2302-7
7. Wallace J L, Wang R: Hydrogen sulfide-based therapeutics: exploiting a unique but ubiquitous gasotransmitter. Nat Rev Drug Discov 2015; 14: 329-45
8. Kimura H: Signalling by hydrogen sulfide and polysulfides via protein S-sulfuration. Br J Pharmacol 2020; 177: 720-33
9. Ida T, Sawa T, Ihara H, Tsuchiya Y, Watanabe Y, Kumagai Y, Suematsu M, Motohashi H, Fujii S, Matsunaga T, Yamamoto M, Ono K, Devarie-Baez N O, Xian M, Fukuto J M, Akaike T: Reactive cysteine persulfides and S-polythiolation regulate oxidative stress and redox signaling. Proc Natl Acad Sci USA 2014; 111:7606-11
10. Sawa T, Motohashi H, Ihara H. Akaike T: Enzymatic Regulation and Biological Functions of Reactive Cysteine Persulfides and Polysulfides. Biomolecules 2020: 10: 1245
11. Zhang T, Ono K, Tsutsuki H. Ihara H, Islam W, Akaike T, Sawa T: Enhanced Cellular Polysulfides Negatively Regulate TLR4 Signaling and Mitigate Lethal Endotoxin Shock. Cell Chem Biol 2019; 26: 686-98.e4
12. Barayeu U, Schilling D, Eid M, Xavier da Silva T N, Schlicker L, Mitreska N, Zapp C, Grater F, Miller A K, Kappl R, Schulze A, Friedmann Angeli J P, Dick T P: Hydropersulfides inhibit lipid peroxidation and ferroptosis by scavenging radicals. Nat Chem Biol 2022. https://doi.org/10.1038/s41589-022-01145-w
13. Kimura H: Signaling molecules: hydrogen sulfide and polysulfide. Antioxid Redox Signal 2015; 22: 362-76
14. Braunstein I, Engelman R, Yitzhaki O, Ziv T, Galardon E. Benhar M: Opposing effects of polysulfides and thioredoxin on apoptosis through caspase persulfidation. J Biol Chem 2020; 295: 3590-600
15. Kakinohana M, Marutani E, Tokuda K, Kida K, Kosugi S, Kasamatsu S, Magliocca A, Ikeda K, Kai S, Sakaguchi M, Hirai S, Xian M, Kaneki M, Ichinose F: Breathing hydrogen sulfide prevents delayed paraplegia in mice. Free Radic Biol Med 2019; 131: 243-50
16. Wintner E A, Deckwerth T L, Langston W, Bengtsson A, Leviten D, Hill P, Insko M A, Dumpit R, VandenEkart E, Toombs C F, Szabo C: A monobromobimane-based assay to measure the pharmacokinetic profile of reactive sulphide species in blood. Br J Pharmacol 2010; 160: 941-57
17. Bianco C L, Akaike T, Ida T, Nagy P, Bogdandi V, Toscano J P, Kumagai Y, Henderson C F, Goddu R N, Lin J. Fukuto J M: The reaction of hydrogen sulfide with disulfides: formation of a stable trisulfide and implications for biological systems. Br J Pharmacol 2019; 176: 671-683
18. Kunikata H, Ida T, Sato K, Aizawa N, Sawa T, Tawarayama H, MurayamaN, Fujii S, Akaike T, Nakazawa T: Metabolomic profiling of reactive persulfides and polysulfides in the aqueous and vitreous humors. Sci Rep 2017; 7: 41984
19. Numakura T, Sugiura H, Akaike T, Ida T, Fujii S, Koarai A, Yamada M, Onodera K, Hashimoto Y, Tanaka R, Sato K, Shishikura Y, Hirano T, Yanagisawa S, Fujino N, Okazaki T, Tamada T, Hoshikawa Y, Okada Y, Ichinose M: Production of reactive persulfide species in chronic obstructive pulmonary disease. Thorax 2017; 72: 1074-83
20. Aquilano K, Baldelli S, Ciriolo M R: Glutathione: new roles in redox signaling for an old antioxidant. Front Pharmacol 2014; 5: 196
21. Yabuki Y, Fukunaga K: Oral administration of glutathione improves memory deficits following transient brain ischemia by reducing brain oxidative stress. Neuroscience 2013; 250: 394-407
22. Sadhu S S, Xie J, Zhang H, Perumal O, Guan X: Glutathione Disulfide Liposomes—a Research Tool for the Study of Glutathione Disulfide Associated Functions and Dysfunctions. Biochem Biophys Rep 2016; 7: 225-229
23. Giustarini D, Rossi R, Milzani A, Colombo R, Dalle-Donne I: S-glutathionylation: from redox regulation of protein functions to human diseases. J Cell Mol Med 2004; 8: 201-12
24. Xiong Y, Uys J D, Tew K D, Townsend D M: S-glutathionylation: from molecular mechanisms to health outcomes. Antioxid Redox Signal 2011; 15: 233-70
25. Toth F, Cseh E K, Vecsei L: Natural Molecules and Neuroprotection: Kynurenic Acid, Pantethine and α-Lipoic Acid. Int J Mol Sci 2021; 22: 403
26. Brunetti D, Dusi S, Giordano C, Lamperti C. Morbin M. Fugnanesi V, Marchet S, Fagiolari G, Sibon O, Moggio M, d'Amati G, Tiranti V: Pantethine treatment is effective in recovering the disease phenotype induced by ketogenic diet in a pantothenate kinase-associated neurodegeneration mouse model. Brain 2014; 137: 57-68
27. Baranger K, van Gijsel-Bonnello M, Stephan D, Carpentier W, Rivera S, Khrestchatisky M, Gharib B, De Reggi M, Benech P: Long-Term Pantethine Treatment Counteracts Pathologic Gene Dysregulation and Decreases Alzheimer's Disease Pathogenesis in a Transgenic Mouse Model. Neurotherapeutics 2019; 16: 1237-1254
28. Smith P D, Puskas F, Meng X, Cho D, Cleveland J C, Jr., Weyant M J, Watkins M T, Fullerton D A, Reece T B: Ischemic dose-response in the spinal cord: both immediate and delayed paraplegia. J Surg Res 2012; 174: 238-44
29. Smith P D, Puskas F, Meng X, Lee J H, Cleveland J C, Jr., Weyant M J, Fullerton D A, Reece T B: The evolution of chemokine release supports a bimodal mechanism of spinal cord ischemia and reperfusion injury. Circulation 2012; 126: S110-7
30. Thome R G, Pronk G J, Padmanabhan V, Frey W H, 2nd: Delivery of insulin-like growth factor-I to the rat brain and spinal cord along olfactory and trigeminal pathways following intranasal administration. Neuroscience 2004; 127: 481-96
31. Rossi F, Perale G, Papa S, Forloni G, Veglianese P: Current options for drug delivery to the spinal cord. Expert Opin Drug Deliv 2013; 10: 385-96
32. Bartanusz V, Jezova D, Alajajian B, Digicaylioglu M: The blood-spinal cord barrier: morphology and clinical implications. Ann Neurol 2011; 70: 194-206
33. Awad H, Ankeny D P, Guan Z, Wei P, McTigue D M, Popovich P G: A mouse model of ischemic spinal cord injury with delayed paralysis caused by aortic cross-clamping. Anesthesiology 2010; 113: 880-91
34. Basso D M, Fisher L C, Anderson A J, Jakeman L B, McTigue D M, Popovich P G: Basso Mouse Scale for locomotion detects differences in recovery after spinal cord injury in five common mouse strains. J Neurotrauma 2006; 23: 635-59
35. Takata T, Jung M, Matsunaga T, Ida T, Morita M, Motohashi H, Shen X, Kevil C G, Fukuto J M, Akaike T: Methods in sulfide and persulfide research. Nitric Oxide 2021; 116: 47-64
36. Hamid H A, Tanaka A, Ida T, Nishimura A, Matsunaga T, Fujii S, Morita M, Sawa T, Fukuto J M, Nagy P, Tsutsumi R, Motohashi H, Ihara H, Akaike T: Polysulfide stabilization by tyrosine and hydroxyphenyl-containing derivatives that is important for a reactive sulfur metabolomics analysis. Redox Biol 2019; 21: 101096
37. Kasamatsu S, Ida T, Koga T, Asada K, Motohashi H, Ihara H, Akaike T: High-Precision Sulfur Metabolomics Innovated by a New Specific Probe for Trapping Reactive Sulfur Species. Antioxid Redox Signal 2021; 34: 1407-1419
38. Takano Y, Hanaoka K, Shimamoto K, Miyamoto R, Komatsu T, Ueno T, Terai T, Kimura H, Nagano T, Urano Y: Development of a reversible fluorescent probe for reactive sulfur species, sulfane sulfur, and its biological application. Chem Commun (Camb) 2017; 53: 1064-7
39. Marutani E, Kosugi S, Tokuda K, Khatri A, Nguyen R, Atochin D N, Kida K, Van Leyen K, Arai K, Ichinose F: A novel hydrogen sulfide-releasing N-methyl-D-aspartate receptor antagonist prevents ischemic neuronal death. J Biol Chem 2012; 287: 32124-35
40. Faul F, Erdfelder E, Lang A G, Buchner A: G*Power 3: a flexible statistical power analysis program for the social, behavioral, and biomedical sciences. Behav Res Methods 2007; 39: 175-91
41. Switzer C H, Guttzeit S, Eykyn T R, Eaton P: Cysteine trisulfide oxidizes protein thiols and induces electrophilic stress in human cells. Redox Biol 2021: 47: 102155
42. Gao Y, Fu R, Wang J, Yang X, Wen L, Feng J: Resveratrol mitigates the oxidative stress mediated by hypoxic-ischemic brain injury in neonatal rats via Nrf2/HO-1 pathway. Pharm Biol 2018; 56: 440-449
43. Bell M T, Puskas F, Agoston V A, Cleveland J C, Jr., Freeman K A, Gamboni F, Herson P S, Meng X, Smith P D, Weyant M J, Fullerton D A, Reece T B: Toll-like receptor 4-dependent microglial activation mediates spinal cord ischemia-reperfusion injury. Circulation 2013; 128: S152-6
44. Ito D, Tanaka K, Suzuki S, Dembo T, Fukuuchi Y: Enhanced expression of Iba1, ionized calcium-binding adapter molecule 1, after transient focal cerebral ischemia in rat brain. Stroke 2001; 32: 1208-15
45. Marutani E, Sakaguchi M, Chen W, Sasakura K, Liu J, Xian M, Hanaoka K, Nagano T, Ichinose F: Cytoprotective effects of hydrogen sulfide-releasing N-methyl-D-aspartate receptor antagonists are mediated by intracellular sulfane sulfur. Medchemcomm 2014; 5: 1577-83
46. Marutani E, Yamada M, Ida T, Tokuda K, Ikeda K, Kai S, Shirozu K, Hayashida K, Kosugi S, Hanaoka K. Kaneki M, Akaike T, Ichinose F: Thiosulfate Mediates Cytoprotective Effects of Hydrogen Sulfide Against Neuronal Ischemia. J Am Heart Assoc 2015; 4: e002125
47. Song J, Park J, Oh Y, Lee J E: Glutathione suppresses cerebral infarct volume and cell death after ischemic injury: involvement of FOXO3 inactivation and Bcl2 expression. Oxid Med Cell Longev 2015; 2015: 426069
48. Rassy D, Bárcena B, Pérez-Osorio I N, Espinosa A, Peón A N, Terrazas L I, Meneses G, Besedovsky H O, Fragoso G, Sciutto E: Intranasal Methylprednisolone Effectively Reduces Neuroinflammation in Mice With Experimental Autoimmune Encephalitis. J Neuropathol Exp Neurol 2020; 79: 226-37
49. Hanson L R, Frey W H, 2nd: Intranasal delivery bypasses the blood-brain barrier to target therapeutic agents to the central nervous system and treat neurodegenerative disease. BMC Neurosci 2008; 9 Suppl 3: S₅
50. Tokuda K, Kida K, Marutani E, et al. Inhaled Hydrogen Sulfide Prevents Endotoxin-Induced Systemic Inflammation and Improves Survival by Altering Sulfide Metabolism in Mice. Antioxidants & Redox Signaling 2012; 17:11-21.
51. Marutani E, Morita M, Hirai S, et al. Sulfide catabolism ameliorates hypoxic brain injury. Nat Commun 2021; 12:3108.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for the treatment, or reduction of risk, of a disorder associated with neurodegeneration in a subject, the method comprising nasally administering a therapeutically or prophylactically effective amount of a composition comprising Glutathione Trisulfide (GSSSG), pantethine trisulfide (PTN-SSS), or lipoic acid trisulfide (LA-SSS) to a subject in need thereof.

2. The method of claim 1, further wherein the composition comprising GSSSG is prepared by dissolving a crystalline form of GSSSG in buffered saline at pH 3-6.

3. The method of claim 1, wherein the disorder is post-ischemic neuronal death.

4. The method of claim 1, wherein the disorder is a chronic cerebral degenerative disease.

5. The method of claim 1, wherein the chronic cerebral degenerative disease is multi-infarct dementia, Alzheimer's disease, Parkinson's disease, or Lewy body dementia.

6. The method of claim 1, comprising administering an effective amount of a composition comprising GSSSG, PTN-SSS, or LA-SSS within a few minutes to hours after a traumatic injury occurs.

7. The method of claim 1, comprising administering an effective amount of a composition comprising GSSSG, PTN-SSS, or LA-SSS before a scheduled thoracic and/or abdominal aortic surgical procedure.

8. The method of claim 7, comprising administering an effective amount of a composition comprising GSSSG, PTN-SSS, or LA-SSS hours to days before a scheduled thoracic and/or abdominal aortic surgical procedure.

9. The method of claim 8, comprising administering an effective amount of a composition comprising GSSSG, PTN-SSS, or LA-SSS 2-24 hours, and/or 1, 2, 3, 4, 5, 6, and/or 7 days before the scheduled thoracic and/or abdominal aortic surgical procedure.

10. A composition for nasal administration comprising Glutathione Trisulfide (GSSSG), pantethine trisulfide (PTN-SSS), or lipoic acid trisulfide (LA-SSS) for use in the treatment, or reduction of risk, of a disorder associated with neurodegeneration in a subject, the method comprising nasally administering a therapeutically or prophylactically effective amount of to a subject in need thereof.

11. The composition for the use of claim 10, wherein the composition is prepared by a method comprising dissolving a crystalline form of GSSSG in buffered saline at pH 3-6.

12. The composition for the use of claim 10, wherein the disorder is post-ischemic neuronal death.

13. The composition for the use of claim 10, wherein the disorder is a chronic cerebral degenerative disease.

14. The composition for the use of claim 10, wherein the chronic cerebral degenerative disease is multi-infarct dementia, Alzheimer's disease, Parkinson's disease, or Lewy body dementia.

15. The composition for the use of claim 10, wherein the method comprises administering an effective amount of a composition comprising GSSSG, PTN-SSS, or LA-SSS within a few minutes to hours after a traumatic injury occurs.

16. The composition for the use of claim 10, wherein the method comprises administering an effective amount of a composition comprising GSSSG, PTN-SSS, or LA-SSS before a scheduled thoracic and/or abdominal aortic surgical procedure.

17. The composition for the use of claim 16, wherein the method comprises administering an effective amount of a composition comprising GSSSG, PTN-SSS, or LA-SSS hours to days before a scheduled thoracic and/or abdominal aortic surgical procedure.

18. The composition for the use of claim 17, wherein the method comprises administering an effective amount of a composition comprising GSSSG, PTN-SSS, or LA-SSS 2-24 hours, and/or 1, 2, 3, 4, 5, 6, and/or 7 days before the scheduled thoracic and/or abdominal aortic surgical procedure.

19. A device for nasal administration of a composition comprising Glutathione Trisulfide (GSSSG), pantethine trisulfide (PTN-SSS), or lipoic acid trisulfide (LA-SSS).

20. The device of claim 19, which is a nebulizer or a pressured container or aerosol dispenser further comprising a propellant.
